(19) United States
(12) Patent Application Publication
(10) Pub. No.: US 2010/0129465 A1
(43) Pub. Date: May 27, 2010

(54) Methods and Compositions Related to Acne Treatment

(76) Inventors: Roger Blotsky, Goodyear, AZ (US); Ramon Figueroa, Miami, FL (US); Leslie Blodgett, San Francisco, CA (US)

Correspondence Address: Ballard Spahr LLP, SUITE 1000, 999 PEACHTREE STREET, ATLANTA, GA, 30309-3915, US

(21) Appl. No.: 12/497,387

(22) Filed: Jul. 2, 2009

Related U.S. Application Data
(60) Provisional application No. 61/078,121, filed on Jul. 3, 2008.

(51) Int. Cl. A61K 33/42; A61P 17/10

(52) U.S. Cl. 424/602

(57) ABSTRACT

Disclosed herein are methods and compositions for treating acne using a composition comprising a large amount of naturally occurring minerals.

### PRIORITY INFORMATION

This application claims priority to U.S. Provisional Application 61/078,121, filed Jul. 2, 2008. This application is hereby incorporated by reference in its entirety.

### BACKGROUND OF THE INVENTION

This invention relates to the treatment of acne vulgaris, commonly known simply as “acne.” Acne is a disease of the skin in which the pilosebaceous structures of the skin become inflamed, leading to the formation of comedones, pustules and nodules. Acne can lead to permanent scarring in severe cases.

It is generally believed that acne arises when hyperkeratosis of the pilosebaceous structure wholly or partially blocks the opening of the structure, resulting in comedones filled with sebum, keratin, and Propionibacterium acnes. These lesions are commonly identified as acne. P. acnes naturally occurs in normal skin, but is especially and characteristically present in acne lesions. It is believed that metabolic byproducts and waste from P. acnes within the pilosebaceous structures cause or contribute to the inflammation of acne lesions.

Conventional acne treatments have taken many forms. Topical keratolytic agents, such as salicylic acid are sometimes used. Keratolytic agents are thought to encourage the opening up of blocked pilosebaceous structures, thereby reducing conditions that are favorable to inflammation. Benzoyl peroxide, an anti-microbial, remains a popular and effective treatment. Topical antibiotics, such as clindamycin, which are effective against P. acnes, have also been used with a view towards preventing the formation of metabolic byproducts from this organism. Topical retinoids such as tretinoin have also been used in the treatment of acne.

Systemic (i.e. non-topical) treatments for acne include the use of oral antibiotics in more serious cases. These treatments are directed towards the reduction in the amount P. acnes in the skin, especially the pilosebaceous structures, and seek to reduce the inflammation caused by waste materials and metabolic byproducts from these organisms. Tetracycline antibiotics are most commonly used for this purpose. These include tetracycline, minocycline and doxycycline. Erythromycinis also sometimes used.

Standard oral minocycline therapy for acne in pediatric patients calls for the administration of a 4 mg/kg initial loading dose, and a 2 mg/kg dose every 12 hours thereafter. This results in a dose of 6 mg/kg on the first day of treatment and a 4 mg/kg dose each day thereafter. In adults, a 200 mg initial dose is followed by a 100 mg dose every 12 hours thereafter. In a typical patient, this results in about a 4.5 mg/kg dose on the first day of treatment, and 3.0 mg/kg dose each day thereafter.

In cases where acne does not respond to oral antibiotic treatment, oral isotretinoin is sometimes used. While effective, isotretinoin is also powerfully teratogenic, and women of childbearing age are required to use multiple methods of contraception while taking the drug.

Mineral elements are essential to life. The body however does not manufacture a single mineral element although all tissue and internal fluids contain them from bones, teeth, soft tissue, muscle, blood and nerve cells. The usefulness of mineral elements and of trace mineral elements in biological systems has been scientifically and medically established. Their complimentary function for enhancing nutrient exchange, improved conductivity of cellular transport, support essential osmotic balance of every tissue, fluid, cell and organ, and play a role on everything from muscle response, to transmission of messages through the nervous system, the production of hormones, digestion, and utilization of nutrients. They play a significant role in disease prevention not only in the functions described above, but on a genetic fundamental level, as biological systems require mineral elements to effectively and accurately program DNA synthesis required for cell replication. Any defective programming in DNA synthesis by deficient mineral element can lead to abnormal replication and alternatively promote disease state or death.

The presence in the body of many mineral elements is the result of supplementation through diet. Macro mineral elements are those that the body requires in greater quantities than 100 mg daily, while Micro mineral elements are those that the body requires less than 100 mg daily. Food consumption, particularly of fruits and vegetables, is the only means to supplement vital mineral elements to the body. The introduction of processed food and the insurgence of soil mineral depletion have created a food market less apt to derive and deliver the mineral element requirements that were once delivered only by consumption. Today's synthetic vitamin and mineral element supplement market (which is valued in the billions of dollars) has been established on the basis that the human body is not getting all the necessary mineral elements through normal food consumption.

Soil depletion phenomena are real and measurable. Restoration of soil involves methods of crop re-cycling and use or organic fertilizers to help reconstitute the mineral content of soil. The use of organic fertilizers has been increasing in usage over the last three decades. Their increased usage is the result of environmental and agricultural concerns for moving towards a chemical-free and pesticide-free method of crop production coupled with a means for replenishment that can alleviate the soil depletion of minerals on farms overburdened by decades of use.

Soil taxonomy and the many sub-classifications yield earth matter that collectively includes all known natural minerals. Soils vary in their mineral content with some having predominant concentrations of certain minerals and trace minerals. The minerals can be concentrated from the soil using extraction techniques known in the art and are usually identified and quantified by analytical equipment.

In all cases, soil classification and the extraction techniques applied to capture or recover minerals are the limiting factors in maximizing the total number and amounts of minerals identified and quantified. Most extraction techniques fail to capture a wide spectrum of inherent minerals found in soil.

### SUMMARY

Disclosed herein is a method of producing a composition for treating acne, the method comprising: combining at least two components of each individual phase, wherein the components of Phase A are:
(a) bismuth oxychloride,
(b) mica with magnesium myristate,
(c) mica with iron oxide and barium sulfate,
(d) corn starch modified,
(e) dimethicone/vinyl dimethicone crosspolymer with silicate,
(f) illite,
(g) caolinitic mineral clay, and
(h) zinc estrum;
the components of Phase B are:
(a) kaolin,
(b) silica,
(c) soil mineral concentrate, and
(d) calcium sodium phosphosilicate with mica;
the components of Phase C are:
(a) tapioca starch, and
(b) melaleuca alterniflolia extract;
the components of Phase D are:
(a) sulfur precipitated, USP,
(b) amorphous fumed silicone dioxide,
(c) farnesol, maltodextrin, dextrin,
(d) aloe barbadensis juice in powder form,
(e) algae extract, and
(f) niacinamide;
and the components of Phase E are:
(a) phyllanthus emblica fruit extract,
(b) melaleuca altenifolia leaf oil and cyclodextrin,
(c) sephora angustifolia root extract,
(d) betaine salicylate,
(e) white willow bark, and
(f) cyclodextrin/retinol;
and once at least two components of each individual phase are mixed together, then combining all of Phases A-E together, thereby producing a composition for treating acne.

Also disclosed are compositions comprising at least two components of each individual phase, wherein the components of Phase A are:
(a) bismuth oxychloride,
(b) mica with magnesium myristate,
(c) mica with iron oxide and barium sulfate,
(d) corn starch modified,
(e) dimethicone/vinyl dimethicone crosspolymer with silicate,
(f) illite,
(g) caolinitic mineral clay, and
(h) zinc estrum;
the components of Phase B are:
(a) kaolin,
(b) silica,
(c) soil mineral concentrate, and
(d) calcium sodium phosphosilicate with mica;
the components of Phase C are:
(a) tapioca starch, and
(b) melaleuca alterniflolia extract;
the components of Phase D are:
(a) sulfur precipitated, USP,
(b) amorphous fumed silicone dioxide,
(c) farnesol, maltodextrin, dextrin,
(d) aloe barbadensis juice in powder form,
(e) algae extract, and
(f) niacinamide;
and the components of Phase E are:
(a) phyllanthus emblica fruit extract,
(b) melaleuca altenifolia leaf oil and cyclodextrin,
(c) sephora angustifolia root extract,
(d) betaine salicylate,
(e) white willow bark, and
(f) cyclodextrin/retinol.

Also disclosed is a method of treating a subject with acne, the method comprising administering to the subject a composition comprising at least two components of each individual phase, wherein the components of Phase A are:
(a) bismuth oxychloride,
(b) mica with magnesium myristate,
(c) mica with iron oxide and barium sulfate,
(d) corn starch modified,
(e) dimethicone/vinyl dimethicone crosspolymer with silicate,
(f) illite,
(g) caolinitic mineral clay, and
(h) zinc estrum;
the components of Phase B are:
(a) kaolin,
(b) silica,
(c) soil mineral concentrate, and
(d) calcium sodium phosphosilicate with mica;
the components of Phase C are:
(a) tapioca starch, and
(b) melaleuca alterniflolia extract;
the components of Phase D are:
(a) sulfur precipitated, USP,
(b) amorphous fumed silicone dioxide,
(c) farnesol, maltodextrin, dextrin,
(d) aloe barbadensis juice in powder form,
(e) algae extract, and
(f) niacinamide;
and the components of Phase E are:
(a) phyllanthus emblica fruit extract,
(b) melaleuca altenifolia leaf oil and cyclodextrin,
(c) sephora angustifolia root extract,
(d) betaine salicylate,
(e) white willow bark, and
(f) cyclodextrin/retinol.

Also disclosed herein are kits comprising at least two components of each individual phase, wherein the components of Phase A are:
(a) bismuth oxychloride,
(b) mica with magnesium myristate,
(c) mica with iron oxide and barium sulfate,
(d) corn starch modified,
(e) dimethicone/vinyl dimethicone crosspolymer with silicate,
(f) illite,
(g) caolinitic mineral clay, and
(h) zinc estrum;
the components of Phase B are:
(a) kaolin,
(b) silica,
(c) soil mineral concentrate, and
(d) calcium sodium phosphosilicate with mica;
the components of Phase C are:
(a) tapioca starch, and
(b) melaleuca alterniflolia extract;
the components of Phase D are:
(a) sulfur precipitated, USP,
(b) amorphous fumed silicone dioxide,
(c) farnesol, maltodextrin, dextrin,
(d) aloe barbadensis juice in powder form,
(e) algae extract, and
(f) niacinamide;
and the components of Phase E are:
(a) phyllanthus emblica fruit extract,
(b) melaleuca altenifolia leaf oil and cyclodextrin,
(c) sephora angustifolia root extract,
(d) betaine salicylate,
(e) white willow bark, and
(f) cyclodextrin/retinol.

One facet of the invention pertains to extraction techniques used to gather, isolate, and concentrate specific mineral elements. For example, in U.S. Pat. No. 4,150,093 Kaminsky and U.S. Pat. No. 3,990,885 Baillie describes hot water extraction of tar sands yielding heavy minerals at specific high concentrations of titanium and zirconium.

Clay soil is one of the three principal types of general soil classifications, the other two being sandy soil and loamy soil. Most soils include silt.

The extraction techniques described herein relate in part to specific soils and soil combination compositions having taxonomic classifications including clay soil, sandy soil, and/or clay-sand soil comprising a combination of clay soil and sandy soil. Sandy soil typically is described as silicates. Soils classified as clay soils contain a significant percentage of clay in their composition, typically at least twenty percent by weight.

### DETAILED DESCRIPTION

### Definitions

Chemical element. Any of more than 100 fundamental metallic and nonmetallic substances that consist of atoms of only one kind and that either singly or in combination constitute all matter, most of these substances lighter in weight than and including uranium being found in nature and the rest being produced artificially by causing changes in the atom nucleus.

Clay. A natural or synthetic colloidal lusterless earthy composition that includes tiny sheet-like layered particles of alumina and/or silica that are less than about 0.002 millimeters in size, that is generally plastic when moist, and that, when naturally occurring, includes decomposed igneous and/or metamorphic rocks. Most clays have a pH in the range of about 4.5 to 8.5. Natural and synthetic clays include mineral elements. Clays can, in additional to having particles less than five microns in size, include particles having a size greater than five microns. Leonardite. A soft, loose-textured coal that has low BTU value. Leonardite is a humate, can include up to 70% by weight minerals, can be formed from lignite, can occur naturally as the result of not being heated and pressurized over time to the extent necessary to produce anthracite, lignite, or bituminous coal, and, can include compost as a component.

Mineral. Any naturally occurring chemical element or compound. A mineral has a characteristic crystal structure and chemical composition or range of compositions.

Mineral element. A chemical element that occurs naturally as or in a mineral. A mineral element can be produced using synthetic or manufacturing processes, however, each mineral element does occur naturally as or in a mineral.

Rare earth or rare earth element. Any one of a group of metallic elements with atomic numbers 58 through 71, including cerium, praseodymium, neodymium, promethium, samarium, europium, gadolinium, terbium, dysprosium, holmium, erbium, thulium, ytterbium, and lutetium. In nature, rare earth elements are bound in combination with nonmetallic elements in the form of phosphates, carbonates, fluorides, silicates, and tantalates.

Sand. A loose material consisting of small but easily distinguishable grains usually less than two millimeters in diameter and more than about 0.02 millimeters in diameter, most commonly of quartz, resulting from the disintegration of rocks.

Silt. Unconsolidated or loose sedimentary material whose constituent rock particles are finer than grains of sand and larger than clay particles, specifically, material consisting of mineral soil particles ranging in diameter from about 0.02 to 0.002 millimeters.

It must be noted that, as used in this specification and the appended claims, the singular forms “a,” “an,” and “the” include plural referents unless the context clearly dictates otherwise.

### Description

All patents, patent applications and references included herein are specifically incorporated by reference in their entireties.

It is to be understood, that the foregoing relates only to preferred embodiments of the present invention and that numerous modifications or alterations can be made therein without departing from the spirit and the scope of the invention as set forth in this disclosure.

The present invention is further illustrated by the following examples, which are not to be construed in any way as imposing limitations upon the scope thereof. On the contrary, it is to be clearly understood that resort can be had to various other embodiments, modifications, and equivalents thereof which, after reading the description herein, can indicate themselves to those skilled in the art without departing from the spirit of the present invention and/or the scope of the appended claims. The present invention comprises mineral, cosmetic, pharmaceutical, agricultural, nutraceuticals, and other compositions and methods for producing the same.

As used herein, the topical dosage form includes various dosage forms known in the art such as lotions (an emulsion, liquid dosage form, whereby this dosage form is generally for external application to the skin), lotion augmented (a lotion dosage form that enhances drug delivery, whereby augmentation does not refer to the strength of the drug in the dosage form), gels (a semisolid dosage form that contains a gelling agent to provide stiffness to a solution or a colloidal dispersion, whereby the gel may contain suspended particles), ointments (a semisolid dosage form, usually containing <20% water and volatiles 5 and >50% hydrocarbons, waxes, or polyols as the vehicle, whereby this dosage form is generally for external application to the skin or mucous membranes), ointment augmented (an ointment dosage form that enhances drug delivery, whereby augmentation does not refer to the strength of the drug in the dosage form), creams (an emulsion, semisolid dosage form, usually containing >20% water and volatiles 5 and/or <50% hydrocarbons, waxes, or polyols as the vehicle, whereby this dosage form is generally for external application to the skin or mucous membranes), cream augmented (a cream dosage form that enhances drug delivery, whereby augmentation does not refer to the strength of the drug in the dosage form), emulsion (a dosage form consisting of a two-phase system comprised of at least two immiscible liquids, one of which is dispersed as droplets, internal or dispersed phase, within the other liquid, external or continuous phase, generally stabilized with one or more emulsifying agents, whereby emulsion is used as a dosage form term unless a more specific term is applicable, e.g. cream, lotion, ointment), suspensions (a liquid dosage form that contains solid particles dispersed in a liquid vehicle), suspension extended release (a liquid preparation consisting of solid particles dispersed throughout a liquid phase in which the particles are not soluble; the suspension has been formulated in a manner to allow at least a reduction in dosing frequency as compared to that drug presented as a conventional dosage form, e.g., as a solution or a prompt drug-releasing, conventional solid dosage form), pastes (A semisolid dosage form, containing a large proportion, 20-50%, of solids finely dispersed in a fatty vehicle, whereby this dosage form is generally for external application to the skin or mucous membranes), solutions (a clear, homogeneous liquid dosage form that contains one or more chemical substances dissolved in a solvent or mixture of mutually miscible solvents), powders (as discussed herein), shampoos (a lotion dosage form which has a soap or detergent that is usually used to clean the hair and scalp; it is often used as a vehicle for dermatologic agents), shampoo suspensions (a liquid soap or detergent containing one or more solid, insoluble substances dispersed in a liquid vehicle that is used to clean the hair and scalp and is often used as a vehicle for dermatologic agents), aerosol foams (i.e., a dosage form containing one or more active ingredients, surfactants, aqueous or nonaqueous liquids, and the propellants; if the propellant is in the internal discontinuous phase, i.e., of the oil-in-water type, a stable foam is discharged, and if the propellant is in the external continuous phase, i.e., of the water-in-oil type, a spray or a quick-breaking foam is discharged), sprays (a liquid minutely divided as by a jet of air or steam), metered spray (a non-pressurized dosage form consisting of valves which allow the dispensing of a specified quantity of spray upon each activation), suspension spray (a liquid preparation containing solid particles dispersed in a liquid vehicle and in the form of coarse droplets or as finely divided solids to be applied locally, most usually to the nasal-pharyngeal tract, or topically to the skin), jellies (a class of gels, which are semisolid systems that consist of suspensions made up of either small inorganic particles or large organic molecules interpenetrated by a liquid—in which the structural coherent matrix contains a high portion of liquid, usually water), films (a thin layer or coating), film extended release (a drug delivery system in the form of a film that releases the drug over an extended period in such a way as to maintain constant drug levels in the blood or target tissue), film soluble (a thin layer or coating which is susceptible to being dissolved when in contact with a liquid), sponges (a porous, interlacing, absorbent material that contains a drug, whereby it is typically used for applying or introducing medication, or for cleansing, and whereby a sponge usually retains its shape), swabs (a small piece of relatively flat absorbent material that contains a drug, whereby a swab may also be attached to one end of a small stick, and whereby a swab is typically used for applying medication or for cleansing), patches (a drug delivery system that often contains an adhesive backing that is usually applied to an external site on the body, whereby its ingredients either passively diffuse from, or are actively transported from, some portion of the patch, whereby depending upon the patch, the ingredients are either delivered to the outer surface of the body or into the body, and whereby a patch is sometimes synonymous with the terms ‘extended release film’ and ‘system’), patch extended release (a drug delivery system in the form of a patch that releases the drug in such a manner that a reduction in dosing frequency compared to that drug presented as a conventional dosage form, e.g., a solution or a prompt drug-releasing, conventional solid dosage form), patch extended release electronically controlled (a drug delivery system in the form of a patch which is controlled by an electric current that releases the drug in such a manner that a reduction in dosing frequency compared to that drug presented as a conventional dosage form, e.g., a solution or a prompt drug-releasing, conventional solid dosage form), and the like. The various topical dosage forms may also be formulated as immediate release, controlled release, sustained release, or the like.

The topical dosage form composition contains an active pharmaceutical ingredient and one or more inactive pharmaceutical ingredients such as excipients, colorants, pigments, additives, fillers, emollients, surfactants (e.g., anionic, cationic, amphoteric and nonionic), penetration enhancers (e.g., alcohols, fatty alcohols, fatty acids, fatty acid esters and polyols), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

Additional ingredients or agents can also be included in the treatment method of the present invention, which can be selected from, but not limited to skin penetration enhancers, skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, solubilizers, anesthetics, colorants, perfumes, preservatives, seeds, broken seed nut shells, silica, clays, beads, luffa particles, polyethylene balls, mica, pH adjusters, processing aids, and combinations thereof.

The disclosed methods pertain to a method for producing compositions including an unusually large number of naturally occurring minerals.

The disclosed compositions pertain to a mineral composition that has an unusually low pH but that does not irritate dermal tissues when applied thereto.

Furthermore, the disclosed compositions pertain to nutritional, cosmetic, and pharmaceutical compositions that include a significant number of mineral elements and that facilitate delivery of the minerals into the body of a human being or animal.

Soil includes very coarse, coarse, fine, very fine, and medium size particle sizes. The coarse particles range in size from 0.5-1.0 mm. The fine particles are from about 0.10 mm to 0.25 mm in size. The medium particles are from 0.25-0.50 mm in size. Very coarse particles are greater than about 1.0 mm in size. The very fine particles are less than about 0.10 mm in size.

The percent sand in clay-sand soil typically by definition equals or is greater than 20% by weight. The percent of silt in clay-sand soil typically by definition equals or is greater than 20% by weight.

Two samples of selected soil were analyzed by A&L laboratories in Memphis, Tenn. with the following results:

| Soil Sample | Classification | % Clay | % Sand | % Silt |
|---|---|---|---|---|
| Site # 4 | Clay | 22.5 | 36.5 | 40.9 |
| Site # 5 | Clay | 23.1 | 24.4 | 52.5 |

The soils from Sites 4 and/or 5 or other sites were collected and subjected to the aqueous extraction process described below to produce both a liquid mineral element composition containing mineral elements and to produce a dry powder mineral element composition. The dry powder mineral element composition is produced by drying the liquid mineral element composition.

Both the liquid mineral element composition and the dry powder mineral element composition capture and recover similar mineral elements to constitute a comprehensive mineral composition. Both liquid and dry powder mineral element compositions produced by the procedures described herein preferably, but not necessarily, contain a minimum of 8 macro mineral elements and a minimum of 60 micro mineral elements.

Physical testing and analysis was also conducted on the liquid and dry mineral element compositions. Typical specifications of liquid extract solution range in color but preferably are from yellow to amber brown and contain between 1 to 10% by weight of mineral elements, most preferably 3-5%. The solution is acidic with a pH ranging from 2.5-4.5, most preferably from 2.5-3.5. The liquid extract can be dried to produce an anhydrous powder. The anhydrous powder presently ranges in color from light-off-white to brown, but preferably from yellow to golden amber, is insoluble in any non-polar solvent such as hydrophobic liquids (oil and fats), is insoluble in alcohol, and is readily soluble, yet non-swelling, in water and hydro-alcoholic solutions at concentrations of 1 to 5%, most preferably at concentrations of 3-5% by weight. The dry powder is partially soluble or capable of being partially suspended in polar solvent in supersaturated solutions. The dry powder can also be easily suspended in non-polar solvents.

As stated above, both liquid and dry mineral element compositions produced by the procedures described herein contain a minimum of 8 macro mineral elements and a minimum of 60 micro mineral elements. The micro mineral elements include trace and rare earth mineral elements.

Compositions of the present invention comprise mineral extract compositions. The method of making such compositions is taught in U.S. Patent Publication No. 2005/0118279, which is herein incorporated in its entirety. Briefly, to make a mineral extract composition of the present invention a soil from a suitable site, comprising the elements as described in U.S. Patent Publication No. 2005/0118279, is collected and subjected to the aqueous extraction process described therein to produce a liquid mineral element composition containing mineral elements which can be dried to produce a dry powder mineral element composition.

Both the liquid mineral extract composition and the dry powder mineral extract composition comprise the mineral elements of a comprehensive mineral composition, as described in U.S. Patent Publication No. 2005/0118279. Both liquid and dry powder mineral extract compositions produced by the procedures described herein generally contain a minimum of 8 macro mineral elements and a minimum of 60 micro mineral elements.

Physical testing and analysis was also conducted on the liquid and dry mineral element compositions. Typical specifications of the liquid mineral extract composition range in color and can be from yellow to amber brown, and contain between 1 to 10% by weight of mineral elements, or from 3-5% by weight of mineral elements. The liquid mineral extract composition is acidic with a pH ranging from 2.5-4.5, or from 2.5-3.5. The liquid mineral extract composition can be dried to produce an anhydrous powder. The anhydrous powder can range in color from light-off-white to brown, or from yellow to golden amber, is insoluble in non-polar solvents such as hydrophobic liquids (oil and fats), is insoluble in alcohol, and is readily soluble, yet non-swelling, in water and hydro-alcoholic solutions at concentrations of 1 to 5% by weight, or at concentrations of 3-5% by weight. The dry powder is partially soluble or capable of being partially suspended in polar solvent in supersaturated solutions.

Both liquid and dry mineral extract compositions produced by the procedures described herein can contain a minimum of 8 macro mineral elements and a minimum of 60 micro mineral elements. The micro mineral elements include trace and rare earth mineral elements.

For example, the dry mineral extract composition can comprise concentrations ranging from 0.0001-20.00% by weight percent, from 0.001%-10%, from 0.1% to 20%, from 1% to 20%, from 1% to 10%, from 5% to 10%, from 10-20%, from 10% to 15%, from 15% to 20%, from 1% to 5%, from 5% to 15%, by weight percent, the macro mineral elements of calcium, chlorine, magnesium, manganese, phosphorous, potassium, silicon, and sodium; and, preferably contain at least sixty micro mineral elements at concentrations ranging from 0.00001-3.0% by weight percent, from 0.0001-1%, from 0.001% to 1%, from 0.01% to 3%, from 0.1% to 3.0%, by weight percent. The micro mineral elements include aluminum, antimony, arsenic, barium, beryllium, bismuth, boron, bromine, cadmium, cerium, cesium, chromium, cobalt, copper, dysprosium, erbium, europium, fluorine, gadolinium, gold, hafnium, holmium, iodine, indium, iridium, iron, lanthanum, lead, lithium, lutetium, mercury, molybdenum, neodymium, nickel, niobium, palladium, platinum, praseodymium, rhenium, rhodium, rubidium, ruthenium, samarium, scandium, selenium, silver, strontium, sulfur, tantalum, terbium, tellurium, thallium, thorium, thulium, tin, titanium, tungsten, vanadium, ytterbium, yttrium, zinc, and zirconium.

The extraction process used to make the mineral extract compositions of the present invention normally does not introduce any minerals as part of the extraction process. Therefore, the source materials, the original clay or other soil that is processed through the extraction method, likely include aluminum silicates and other metal silicates in nature that have been naturally enriched with multiple detectable minerals. If a mineral element is identified and quantified in the aqueous liquid extract, generally, it is identified and quantified in the dry powdered extract in much higher concentrations as a result of drying process or volume reduction.

For example, an extract composition produced using the soil and extractions methods described in U.S. patent application Ser. No. 10/725,729, incorporated herein by reference, was tested by independent analytical testing for conducting chemical analysis using standard techniques of identification and quantification for both dry and liquid forms of the mineral extract composition. The results of testing performed at Teledyne Wah Chang Laboratories in Huntsville, Ala., utilizing scientifically accepted and standard equipment such as titration, inductively coupled plasma, mass spectrometry, and atomic absorption equipment resulted in the mineral element quantification data set forth below in TABLE 1 for an aqueous mineral extract composition and from the dry mineral extract composition that resulted when the aqueous mineral extract composition was dried to produce a powder.

TABLE 1
Mineral Extract Composition

| | | |
|---|---|---|
| | Concentration in aqueous | Concentration |
| Element | liquid composition | in dry powder |

Macro Mineral Elements

| | | | |
|---|---|---|---|
| Calcium | 2900 | ppm | 8% |
| Chlorine | 170 | mg/ml | 0.84%* |
| Magnesium | 460 | ppm | 0.95% |
| Phosphorous | 0.2 | g/L | 0.43% |
| Potassium | 220 | mg/L | 1.2% |
| Silicon | 130 | mg/L | 0.36% |
| Sodium | 720 | mg/L | 2.0% |

Micro Mineral Elements

| Aluminum | 540 | ppm | 0.65% | |
|---|---|---|---|---|
| Antimony | 460 | ppb | 16.0 | ppm |
| Arsenic | 11 | ppm | 3.1 | ppm |
| Barium | 340 | ppb | 11.0 | ppm |
| Beryllium | 0.29 | ppm | .01 | ppm |
| Bismuth | <50 | ppb | <1.00 | ppm |
| Boron | 2.0 | mg/L | 72.0 | ppm |
| Bromine | *Present as part of Chlorine assay | | | |
| Cadmium | <50 | ppb | 1.10 | ppm |
| Total Organic | 12 | g/L | Trace | |
| Carbon | | | | |
| Cerium | 1600 | ppb | 68.00 | ppm |
| Cesium | 82 | ppb | 2.00 | ppm |
| Chromium | 1.8 | ppm | 5.00 | ppm |
| Cobalt | 0.25 | ppm | 1.00 | ppm |
| Copper | 0.09 | ppm | <1.00 | ppm |
| Dysprosium | 230 | ppb | 9.00 | ppm |
| Erbium | 150 | ppb | 6.00 | ppm |
| Europium | <50 | ppb | 2.00 | ppm |
| Fluorine | *Present as part of Chlorine assay | | | |
| Gadolinium | 220 | ppb | 9:00 | ppm |
| Gallium | 70 | ppb | 2.40 | ppm |
| Germanium | <50 | ppb | <1.00 | ppm |
| Gold | <50 | ppm | <1.00 | ppm |
| Hafnium | <0.5 | mg/L | 5.00 | ppm |
| Holmium | <50 | ppb | 2.00 | ppm |
| Iodine | *Present as part of Chlorine assay | | | |
| Indium | <50 | ppb | Trace | |
| Iron | 730 | ppm | 28.00 | ppm |
| Lanthanum | 650 | ppb | 28.00 | ppm |
| Lead | <50 | ppb | <1.00 | ppm |
| Lithium | 0.9 | mg/L | <1.00 | ppm |
| Lutetium | <50 | ppb | <1.00 | ppm |
| Mercury | Trace | | <1.00 | ppm |
| Molybdenum | 3200 | ppb | 120.00 | ppm |
| Neodymium | 1000 | ppb | 45.00 | ppm |
| Nickel | 0.74 | ppm | 2.00 | ppm |
| Niobium | 96 | ppb | 3.00 | ppm |
| Palladium | <500 | ppb | <1.00 | ppm |
| Platinum | <50 | ppb | <1.00 | ppm |
| Praseodymium | 290 | ppb | 10.00 | ppm |
| Rhenium | <50 | ppb | <1.00 | ppm |
| Rhodium | <50 | ppb | <1.00 | ppm |
| Rubidium | 360 | ppb | 11.00 | ppm |
| Ruthenium | <50 | ppb | <1.00 | ppm |
| Samarium | 250 | ppb | 10.00 | ppm |
| Scandium | <400 | ppb | 4.00 | ppm |
| Selenium | 0.63 | mg/L | 21.00 | ppm |
| Silver | <0.02 | ppm | <5.00 | ppm |
| Strontium | 14000 | ppb | 420.00 | ppm |
| Sulfur | 1.1 | g/L | 1.8% | |
| Tantalum | <50 | ppb | <1.00 | ppm |
| Terbium | <50 | ppb | 2.00 | ppm |
| Tellurium | <50 | ppb | <1.00 | ppm |
| Thallium | <50 | ppb | 1.00 | ppm |
| Thorium | 640 | ppm | 22.00 | ppm |
| Thulium | <50 | ppb | 1.00 | ppm |
| Tin | <50 | ppb | <1.00 | ppm |
| Titanium | 9.34 | ppm | 210.00 | ppm |
| Tungsten | 52 | ppb | 17.00 | ppm |
| Vanadium | 4.3 | ppm | 14.00 | ppm |
| Ytterbium | 140 | ppb | 6.00 | ppm |
| Yttrium | 1300 | ppb | 61.00 | ppm |
| Zinc | 1.2 | ppm | 14.00 | ppm |
| Zirconium | 2.0 | mg/L | 62.00 | ppm |

The mineral extract compositions set forth above in Table 1 were produced from naturally occurring soil the analysis of which is reflected below in Table 2.

TABLE 2
Analysis of Naturally Occurring Soil

| | Macro Mineral Elements |
|---|---|
| | Concentration in ppm by weight unless |
| Element | noted as % (for weight percent) |
| Silicon | 25.0% |
| Aluminum | 9.3% |
| Potassium | 4.8% |
| Magnesium | 0.83% |
| Sulfur | 1.6% |
| Iron | 1.6% |
| Calcium | 4.1% |
| Titanium | 0.23% |
| Sodium | 0.138% |
| Manganese | 150 |
| Gallium | 25 |
| Molybdenum | 61 |
| Germanium | 25 |
| Iodine | 7 |
| Bromine | 5.2 |
| Tungsten | 8.1 |
| Hafnium | 2.0 |
| Tantalum | 0.50 |
| Zirconium | 10 |
| Arsenic | 0.2 |
| Antimony | 29 |
| Selenium | 4.1 |
| Zinc | 20 |
| Samarium | 3.5 |
| Holmium | 1.1 |
| Terbium | .62 |
| Iridium | .51 |
| Lutetium | .45 |
| Chromium | 70 |
| Lanthanum | 18 |
| Ruthenium | 7.8 |
| Yttrium | 1.2 |
| Indium | .38 |
| Lead (under) | 17 |
| Niobium | 2.89 |
| Carbon | .19 |
| Hydrogen | .05 |
| Nitrogen | .03 |
| Scandium | 3.7 |
| Cobalt | 4.8 |
| Ytterbium | 1.4 |
| Strontium | 240 |
| Barium | 390 |
| Gold | .68 |
| Europium | .49 |
| Neodymium | 20 |
| Cerium | 40 |
| Cesium | 183 |
| Thorium | Above 100 |
| Uranium | Above 100 |
| Nickel | 60 |
| Beryllium | .10 |
| Bismuth | 14.3 |
| Boron | 7 |
| Cadmium | 1.12 |
| Chloride | 6100 |
| Copper | 2.2 |
| Fluoride | 3.85 |
| Lithium | 1.44 |
| Mercury | 0.166 |
| Palladium | 0.74 |
| Phosphate | 320 |
| Platinum | 0.08 |
| Rhodium | 0.44 |
| Rubidium | 36.5 |
| Silver | 0.3 |
| Tellurium | 0.1 |
| Thulium | 0.65 |
| Tin | 0.44 |
| Vanadium | 8 |
| Dysprosium | 4.0 |
| Praseodymium | 2.0 |
| Thallium | 10 |
| Rhenium | 1.0 |
| Erbium | 2.0 |
| Oxygen | 0.2 |

Once a desirable naturally occurring soil or soil combination is obtained, the soil(s) is subjected to the extraction process described by U.S. patent application Ser. No. 10/725,729. Clay soils, mixtures of clay soils, or mixtures of clay soil(s) and leonardite are preferred in the practice of the disclosed compositions. One reason such soil combinations are preferred is that such soils can be high in the mineral elements deemed important in the practice of the invention. As noted, it is preferred that mineral extract compositions produced in accordance with the disclosed methods and compositions include at least eight macro mineral elements and at least sixty micro mineral elements.

The first step in determining whether a clay soil is acceptable as a source material is to determine of arsenic, lead, mercury, and cadmium are each present in acceptably small concentrations. An aspect of the presently disclosed compositions comprises compositions having the concentration of each of these elements in lower amounts than the concentrations shown below in Table 3.

TABLE 3
Maximum Desired Concentrations of Toxic Elements

| | Maximum Desired Soil Concentration in |
|---|---|
| Element | ppm or ppb |
| Arsenic | 0.2 ppm |
| Lead | 0.17 ppb |
| Mercury | 0.116 ppm |
| Cadmium | 1.12 ppm |

TABLE 4
Preferred Minimum Concentrations of Selected Rare Earth Elements in
Naturally Occurring Soil

| | Preferred Minimum Soil Concentration in |
|---|---|
| Element | ppm |
| Cerium | 40 |
| Praseodymium | 2 |
| Neodymium | 20 |
| Samarium | 3.5 |
| Europium | 0.49 |
| Terbium | 0.62 |
| Dysprosium | 4 |
| Holmium | 1 |
| Erbium | 2 |
| Thulium | 0.65 |
| Ytterbium | 1.2 |
| Lutetium | 0.45 |

The concentration of the elements listed in Table 4 can vary as desired, but, as noted, it is desirable to have at least the concentration of each element as noted in Table 4. Source material soil for composition of the present disclosed compositions can comprise one or all of the rare earth elements listed in Table 4. For example, a lanthanum concentration of at least eighteen ppm and a scandium concentration of at least three and seven-tenths ppm can be found in a source material soil.

Concentrations of promethium and gadolinium can also be found. Source material soil for composition of the disclosed compositions can comprise at least ten rare earth elements, at least twelve, or more rare earth elements and optionally include lanthanum and scandium. Though not wishing to be bound by any particular theory, it is theorized that the presence of rare earth elements in the soil, and in the mineral extract compositions derived from the source material soil, is believed to be useful in improving the efficacy of the mineral extract compositions when ingested or when transdermally absorbed by the body.

The clay soil or soil combination also includes at least 5% by weight calcium, preferably at least 10% by weight calcium, and most preferably at least 20% by weight calcium. Concentrations of calcium of 25% by weight or greater are acceptable.

The clay soil or soil combination also includes at least 5% by weight silica, preferably at least 10% by weight silica, and most preferably at least 20% by weight silica. Concentrations of silica of 25% by weight or greater are acceptable.

The clay soil or soil combination also includes at least 0.25% by weight phosphorous, preferably at least 1% by weight phosphorous, and most preferably at least 2% by weight phosphorous.

Leonardite is a valuable mineral source in producing soils that are subjected to the extraction process illustrated in FIG. 1. Once a clay soil or clay and soil combination is provided or is combined to yield the mineral elements, as shown in FIG. 1 and taught by U.S. patent application Ser. No. 10/725,729. The following example describes the extraction process by way of illustration, and not limitation of the invention.

### Examples

### Example 1

### Extraction Process

In general, the extraction of the source material soil uses the following steps. Water, typically purified using known methods such as reverse osmosis, is added to citric acid and the source material soil in a mixing tank. The amount of citric acid (or of phosphoric acid or other edible acid(s)) or combinations thereof, can be in the range of 0.25% to 7.5% of the weight of water utilized, but typically is in the range of 1.0% to 2.0%. The water, citric acid and source material soil, form a slurry and is gently agitated (for example, with a blade slowly rotating at from one to ten RPM) for about an hour, although the agitation time can vary as desired. The slurry from the tank is directed into a settling tank to permit particulates to settle downwardly out of the slurry. The slurry is maintained in the settling tank for any desired length of time, in the range of about one to ten days. As the length of time that the slurry is maintained in the settling tank increases, the amount of liquid that can be drawn out of the tank and sent to a cooling tank or concentrator increases and the amount of solids that have settled to the bottom of the tank increases. Additives can be used to facilitate the settling of solids from slurry. After the slurry has resided in settling tank for the desired period of time, liquid is drawn out of the tank to a cooling tank, or directly to the concentrator. The solids on the bottom of tank can be reprocessed, discarded, or can be otherwise utilized.

The cooling tank cools the fluid from the settling tank to a temperature in the range of 40-70° F. (5 to 21° C.). Cooled liquid is sent to the concentrator.

The concentrator removes water from the cooled liquid. This can be accomplished using known methods such as a thin film composite reverse osmosis system or evaporation. The resulting concentrate liquid, comprising the minerals extracted from the original slurry, is directed to a cooling tank or to a dryer, depending if storage or further processing is desired. The cooling tank cools the concentrate liquid to 40 to 70° F. (5 to 20° C.) to prevent the growth and yeast and mold.

One preferred reverse osmosis system includes eight hollow tubes or “vessels” that are about four inches in diameter and forty inches long. Each tube houses three concentric cylindrical membranes. The permeability flow rate is approximately 80% to 95% rejection, depending on the feed rate and the concentration of mineral elements in the fluid being treated. The spacing between the three concentric membranes is about ¼ inch. There are three ring couplers and one end plug per tube. The maximum pressure allowed by the cylindrical membranes is about 600 psig. A pressure of between 300 to 450 is recommended and is normally used. The membranes are to be utilized at a temperature of 135 degrees F. (57 degrees C.) or less. The temperature of the fluid and the membrane is, however, typically maintained in the range of 55 degrees F. to 65 degrees F. (12 to 20 degrees C.). The fluid is processed by passing it sequentially through each of the eight tubes.

If desired, concentration systems other than reverse osmosis systems can be utilized. Such other systems are not believed comparable to a reverse osmosis system in terms of cost and efficiency.

In FIG. 1 the “slurry” by product produced by the concentrator comprises clean usable water with a low concentration of mineral elements. The aqueous concentrate liquid produced by the concentrator is directed to cooling or directly to the dryer. The concentrate liquid is cooled to 40 degrees F. to 70 degrees F. (5 degrees to 20 degrees C.) to prevent the growth and yeast and mold.

The concentrate liquid produced by concentrator has a pH of approximately 3. The concentrate liquid typically includes from three to twelve percent by weight mineral elements, i.e. if the mineral elements are separated from the concentrate liquid, a dry material is produced that has a weight equaling about 3% to 12% by weight of the concentrate liquid. The pH of the concentrate liquid is adjusted by varying the amount of citric acid or other edible acid and/or alkaline or acidic soil added to the mixing tank and is in the range of pH 2.0 to pH 5.0, preferably pH 2.5 to pH 3.5. The pH of the concentrate liquid (and dry powder or other material produced therefrom) preferably is less than pH 4.5. A mineral extract concentration of at least eight percent can be provided for injection into a dryer. Any desired drying system can be utilized, such as a tower into which the concentrate liquid is sprayed to produce a powder.

Any desired drying system can be utilized. The present drying apparatus consists of a tower into which the concentrate fluid is sprayed. Air in the tower is heated. The concentrate fluid is sprayed in a pattern that causes the spray to swirl down the sides of the tower. As the spray travels down the sides of the tower, the water evaporates, producing powder particles including mineral elements. The powder falls downwardly to the bottom of the tower. Moist air travels upwardly through the center of the tower and is directed 23 to a bag house 22. The moist air enters elongate air-permeable hollow generally cylindrical bags in the bag house. The air travels outwardly through the walls of the bags and leaves behind powder particles on the inside surfaces of the bag. The bags are shaken each thirty seconds to cause the powder on the inner surfaces of the bag to fall downwardly for collection. Table 1 illustrates the mineral element concentration in the powder produced in dryer 15 when the liquid mineral element concentrate having the composition set forth in Table 1 was directed into dryer 15. The dry powder mineral element composition of Table 1 in aqueous solution has a pH of about 3.0.

In one spray system utilized in the dryer 15, the fluid concentrate is directed into dryer 15 under a pressure of about 2500 psi. The orifice size of the spray nozzles utilized is about 0.027 inch. The spray angle of the nozzle is 70 degrees and the average droplet size is about 75 microns.

The use of a nutritional supplement in tablets, soft capsules, bars, processed foods or beverages which contains the small concentrations of the mineral element compositions described herein can be beneficial to health if used to supply sub-toxic dosages of certain mineral elements that can pose a toxic risk. An Acute Oral Toxicity animal study conducted at Northview Pacific Labs in Hercules, Calif. indicated that acute dosages of 1 gram of dry mineral element composition per kilogram of weight of an individual classified the Comprehensive Mineral Composition posed no toxicity risk to an individual. This qualifies the products produced by the processes of the invention as a unique composition that delivers a substantial natural balance of minerals through oral supplementation in a single or multiple dosages for human and veterinary product consumption.

In the area of topical application and delivery of minerals, there is growing evidence that transdermal delivery can be the best route to deliver therapeutic agents, particularly metal drugs. There is also great interest on skin for being the next frontier for better route of delivery of vitamins and minerals for improved systemic absorption and availability. For example, studies at the Graduate School of Science and Technology at Bond University in Australia demonstrated how the gastro-intestinal tract presents a significant barrier to the efficient absorption of both orally administered and inject-able dietary essential trace minerals. Their studies indicate that presenting trace minerals which can penetrate the dermis permits their slow release from the skin with more efficient (relative to incipient toxicity) systemic delivery. Examples are given of dermal application of copper, zinc, titanium, platinum and gold complexes to treat chronic inflammatory disease. Some of these compounds are also anti-cancer agents. Other studies have demonstrated that skin penetration of minerals follow a pattern of organ distribution.

The mineral element compositions described herein can be an ideal multi-mineral product for delivery through the skin qualifying as a unique composition that delivers a substantial natural balance of minerals to the surface of the skin or on stratum corneum for transdermal supplementation. A single or multiple dosage for human and veterinary product application onto the skin can contain small concentrations of the mineral element compositions described herein and can be beneficial to health if used in sub-toxic dosages.

An example of a transdermal product follows.

### Example 2

TABLE 5
Transdermal Mineral Gel Composition

| Ingredients | Weight % |
|---|---|
| Liquid mineral element composition of Table 1 | q.s. to 100% |
| Xanthan gum | 0.30 |
| Diethylene glycol monoethyl ether | 12.00 |
| Ethyl oleate | 2.00 |
| Alcohol SDA 40 | 7.00 |

### Procedure:

Mix xantham gum in liquid mineral element composition using propeller mixer. Add other ingredients one by one.

One Kilogram of Transdermal Mineral Gel Formula Composition delivers onto the stratum corneum no less than 1 ppm of Macro Minerals consisting of a blend of Calcium, Chlorine, Magnesium, Manganese, Phosphorous, Potassium, Silicon, Sodium, and no less that 0.0001 ppm of Micro Minerals consisting of a blend of Aluminum, Antimony, Arsenic, Barium, Beryllium, Bismuth, Boron, Bromine, Cadmium, Cerium, Cesium, Chromium, Cobalt, Copper, Dysprosium, Erbium, Europium, Fluorine, Gadolinium, Gold, Hafnium, Holmium, Iodine, Indium, Iridium, Iron, Lanthanum, Lead, Lithium, Lutetium, Mercury, Molybdenum, Neodymium, Nickel, Niobium, Palladium, Platinum, Praseodymium, Rhenium, Rhodium, Rubidium, Ruthenium, Samarium, Scandium, Selenium, Silver, Strontium, Sulfur, Tantalum, Terbium, Tellurium, Thallium, Thorium, Thulium, Tin, Titanium, Tungsten, Vanadium, Ytterbium, Yttrium, Zinc, Zirconium, to be absorbed and bioavailable selectively by skin as it is delivered transdermally.

The resulting aqueous solutions from the mineral element composition are highly acidic. Preparation of acidic mineral element solutions can useful, particularly for the personal care industry.

### Example 3

Water—100% as supplied through the process of reverse osmosis. pH=5.7.

### Example 4

Water: 95% by weight, as supplied in Example 3 through the process of reverse osmosis. Dry mineral element composition of Table 1: 5% by weight. Mix water and mineral element composition together. The pH of the resulting aqueous solution is 3.0.

The mineral composition aqueous solution of Example 3 is substantially non-irritating to skin and eyes. Acidic solution is normally be irritating to open wounds. For example, aqueous solutions of glycolic acid with a pH=3.0 stings or burns when applied or upon contact to freshly shaven skin. The pH=3.0 aqueous solution of Example 3 causes little or no sting or burning when applied to freshly shaven skin.

The mineral compositions described herein can be an ideal multi-mineral product for delivering exfoliation to the skin.

Skin proliferation, the rate at which cell are born at the basal layer and subsequently shed from the body after reaching the upper layers of the stratum corneum, is an important and dynamic function for maintaining healthy skin. For example, psoriatic patients suffer from proliferation disorders as skin cells do not regenerate or desquamate normally. Because minerals such as zinc and copper play a role in skin proliferation, they have been extensively studied for topical application and have been shown to improve certain skin condition disorder. Skin proliferation disorders such as dandruff have also been studied with the use of minerals to bring about improvements.

Altering the rate of skin proliferation has been the mechanism by which many anti-aging skin care products are promoted. As skin ages the skin proliferation rate decreases, and stimulating cell renewal to a rate that is closer to younger skin has proven to improve the general appearance of skin. Ingredients such as retinoic acids, retinol and alpha hydroxy acids (ABA's) are widely promoted on a global scale for their ability to increase cell turnover and promote younger looking skin.

For these reasons AHA's are a commonly added to skin care products including moisturizers, cleanser, toners, and masks. AHA's are naturally derived from fruit and milk sugars and synthetically made as pharmaceutical and cosmetic acidulant ingredient. They are used in skin care as ‘cosmeceutical’ or functional cosmetic ingredients.

The most commonly used AHA's are glycolic acid and lactic acid. AHA's work mainly as an exfoliant of the skin. They cause the cells of the skin to become “unglued” allowing the dead skin cells at the surface of the skin to slough off, making room for re-growth of new skin. They also indirectly stimulate, through the process of irritation, the production of new cells. They have been reported to improve wrinkling, roughness, and pigmentation on skin after long term application and have been extensively studied.

AHA's as used in skin-care products work best at acidic pH's as it is the free acid and not the neutralized or salt counterparts that have been found effective on the skin as exfoliants. Typically, a pH of 3-5 is optimal when utilizing AHA's. As a result, two major side effects of AHA's are irritation and sun sensitivity. Symptoms of irritation include redness, burning, itching, pain, and possibly scarring. There are milder and other forms of exfoliants on the market today than AHA. Beta hydroxy acids such as salicylic acid have been reported to bring about skin cell turnover rate increases. Retinol (the alcohol form of retinoic acid) has also been extensively used It was unexpectedly found that the liquid mineral element composition and the dry powder mineral element composition produced in accordance with the invention, as well as solutions of the same, were able to cause skin to exfoliate. There appears to be no prior art indicating any anticipatory use of minerals as skin exfoliants or to affect cell renewal.

Topical preparations that included the use of the mineral element compositions of the invention were observed to provide multiple skin benefits. Among the benefits observed was mild exfoliation. Exfoliation was subjectively measured by the ability of skin to be renewed after several days of use, with some mild peeling depending on subject. Skin was observed as less sallow and more translucent. Product containing 5% by weight of the dry powdered mineral element composition of Table 1 in aqueous solution was observed to provide the maximum exfoliation effect.

Typically, AHA products become irritating after several days of use as the skin becomes sensitized to low pH levels of these products. Comparatively, aqueous solutions including 5% by weight of the dry mineral element composition of Table 1 at a pH of 3 demonstrated the ability to exfoliate skin in a non-irritating manner.

It is therefore novel, at least for the mineral element compositions derived by the extraction process described herein, that the mineral element compositions can serve as a new class of cosmetic and dermatological ingredients of exfoliation with significantly less adverse effects such as burning and irritation.

The use of the comprehensive mineral composition in topical over the counter therapeutic products is believed to be beneficial to skin disorders ranging from severe dry skin to treatment of skin disorders. It is known that many macro and micro mineral elements play significant roles in treating skin disorders. For example, copper is essential for production of tyrosinase, an enzyme which is required for the production of melanin for the activation of melanocytes which together with sunscreens protect the skin from UV by initiating tanning. As another example, selenium can help in the treatment and prevention of dandruff and deficiency in the mineral can lead to appearance of premature aging.

The comprehensive mineral compositions described herein can be an ideal multi-mineral product for delivery on the skin qualifying as a unique composition that delivers a substantial natural balance of minerals in a single or multiple dosages for human and veterinary product consumption providing mild exfoliation effects.

### Example 5

TABLE 6
Exfoliant Cleanser Composition

| Ingredient | Weight % |
|---|---|
| Liquid Mineral Element Composition Of | q.s. to 100% |
| Table 1: | |
| TEA Cocoyl Glutamate | 7.00 |
| Glycerin | 5.00 |
| Decyl Glucoside | 5.00 |
| Dimethicone Copolyol Phosphate | 2.00 |
| Preservatives and Fragrance | 1.00 |

### Procedure:

Blend each ingredient one at a time to produce final composition. Apply final composition to skin with or without water, gently rub composition into skin for at least 2 minutes, and rinse with water.

One liter of Exfoliant Cleanser Composition delivers onto the stratum corneum no less than one ppm of Macro Minerals consisting of a blend of Calcium, Chlorine, Magnesium, Manganese, Phosphorous, Potassium, Silicon, Sodium, and no less that 0.0001 ppm of Micro Minerals consisting of a blend of Aluminum, Antimony, Arsenic, Barium, Beryllium, Bismuth, Boron, Bromine, Cadmium, Cerium, Cesium, Chromium, Cobalt, Copper, Dysprosium, Erbium, Europium, Fluorine, Gadolinium, Gold, Hafnium, Holmium, Iodine, Indium, Iridium, Iron, Lanthanum, Lead, Lithium, Lutetium, Mercury, Molybdenum, Neodymium, Nickel, Niobium, Palladium, Platinum, Praseodymium, Rhenium, Rhodium, Rubidium, Ruthenium, Samarium, Scandium, Selenium, Silver, Strontium, Sulfur, Tantalum, Terbium, Tellurium, Thallium, Thorium, Thulium, Tin, Titanium, Tungsten, Vanadium, Ytterbium, Yttrium, Zinc, Zirconium.

### Example 6

TABLE 7
Suspended Minerals Exfoliant Scrub Composition

| | |
|---|---|
| Ingredients | Weight % |

Phase A

| | |
|---|---|
| Water | q.s to 100% |
| Propylene Glycol | 5.00 |

Phase B

| | |
|---|---|
| Hydrogenated Polyisobutene | 10.00 |
| Isopropyl Myristate | 5.00 |
| Mineral Oil | 3.00 |
| PEG 100 Stearate & | 5.00 |
| Glyceryl Monostearate | |
| Polysorbate 20 | 1.00 |
| Beeswax | 2.00 |

Phase C

| | |
|---|---|
| Preservatives and Fragrance | 1.00 |
| Dry Mineral Element | 20.00 |
| Composition of Table 1 | |

### Procedure

Blend ingredients listed above under Phase A and heat to 75 C.

Blend ingredients listed above under Phase B and heat to 77 C.

Add Phase B to Phase A at 77 degrees C. and blend with propeller mixer to produce intermediate composition.

Cool intermediate composition to 40 C., and add ingredients listed above under Phase C to intermediate composition one ingredient at a time to produce final composition.

Cool final composition to 25 C. Apply final composition to skin with or without water, gently rub composition into skin for at least one minute, and rinse with water to exfoliate skin.

One liter of Suspended Minerals Exfoliant Scrub Composition delivers onto the stratum corneum no less than one ppm of Macro Minerals consisting of a blend of Calcium, Chlorine, Magnesium, Manganese, Phosphorous, Potassium, Silicon, Sodium, and no less that 0.0001 ppm of Micro Minerals consisting of a blend of Aluminum, Antimony, Arsenic, Barium, Beryllium, Bismuth, Boron, Bromine, Cadmium, Cerium, Cesium, Chromium, Cobalt, Copper, Dysprosium, Erbium, Europium, Fluorine, Gadolinium, Gold, Hafnium, Holmium, Iodine, Indium, Iridium, Iron, Lanthanum, Lead, Lithium, Lutetium, Mercury, Molybdenum, Neodymium, Nickel, Niobium, Palladium, Platinum, Praseodymium, Rhenium, Rhodium, Rubidium, Ruthenium, Samarium, Scandium, Selenium, Silver, Strontium, Sulfur, Tantalum, Terbium, Tellurium, Thallium, Thorium, Thulium, Tin, Titanium, Tungsten, Vanadium, Ytterbium, Yttrium, Zinc, Zirconium.

### Example 7

TABLE 8
Antibacterial Exfoliant Toner Composition

| Ingredient | Weight Percent |
|---|---|
| Liquid Mineral Element Composition | q.s. to 100% |
| of Table 1 | |
| Hydroxyethyl cellulose | 1.00 |
| Ethyl Alcohol | 62.00 |

### Procedure:

Blend ingredients together one at a time at room temperature to produce the exfoliant toner formula composition. Apply the resulting composition by gently rubbing a small amount into the dermis for about thirty seconds. Then rinse dermis to remove any remaining composition.

One liter of Antibacterial Exfoliant Toner Composition delivers onto the stratum corneum no less than one ppm of Macro Minerals consisting of a blend of Calcium, Chlorine, Magnesium, Manganese, Phosphorous, Potassium, Silicon, Sodium, and no less that 0.0001 ppm of Micro Minerals consisting of a blend of Aluminum, Antimony, Arsenic, Barium, Beryllium, Bismuth, Boron, Bromine, Cadmium, Cerium, Cesium, Chromium, Cobalt, Copper, Dysprosium, Erbium, Europium, Fluorine, Gadolinium, Gold, Hafnium, Holmium, Iodine, Indium, Iridium, Iron, Lanthanum, Lead, Lithium, Lutetium, Mercury, Molybdenum, Neodymium, Nickel, Niobium, Palladium, Platinum, Praseodymium, Rhenium, Rhodium, Rubidium, Ruthenium, Samarium, Scandium, Selenium, Silver, Strontium, Sulfur, Tantalum, Terbium, Tellurium, Thallium, Thorium, Thulium, Tin, Titanium, Tungsten, Vanadium, Ytterbium, Yttrium, Zinc, Zirconium.

Skin is the largest organ of the body. As humans age, skin, like many other organs, gradually loses its ability to function as it once did. It becomes less efficient in all the processes it possesses, and so does its ability to regenerate and repair itself

One skin function that diminishes is based on a reptilian similarity human skin possesses and that is its constant state of renewal. As a result, it is known to those skilled in the field that as skin ages, there is an increase in surface accumulation of dead cells as cell renewal rates decrease.

Skin cell regeneration, a process of replenishing older cells with fresh cell in the constant cycle of renewal, is important in maintaining viable cell health and a healthy youthful appearance. New skin cells are constantly born at deepest basal layer underneath the visible layer by a process of continual cell (keritinocytes) division. As new skin cells mature and nourish the underlying tissue, they proliferate until reaching the visible surface of the skin, eventually losing their nucleus, becoming, flattened and dying while being shed from the body as other new vibrant cells replace them from underneath in a harmonious sequence. The process is often referred to as skin cell renewal, cell turnover, cell proliferation, or cell regeneration.

As humans age, several possible events trigger a decrease in the rate of cell regeneration. Among them are hormonal and other physiological changes that tire the renewal process. It is also possible that weakened blood vessels within the skin and reduced cell surface area limit the nutrient uptake of cells and this slows renewal. It is further possible that the natural cohesion factors that function to release and shed skin loss their efficiency for releasing skin at a sustain pace with the rebirth of new cells, allowing dead skin cells to simply accumulate on the surface.

When cell renewal slows down the visible result of excess accumulation of dead skin cells creates a dull, dry, thicker layer of skin. The excess accumulation of surface skin cells works against the natural glow of fresher cells and accentuates gray skin tones as well as accentuates minor skin imperfections, skin dryness, and accentuation of wrinkles.

Skin exfoliants, representing topically applied products that remove or accelerate the removal of surface skin cells, are commonly used to accelerate the process of surface skin cell renewal. Exfoliant products are known to exist in both chemical and abrasive forms. Dermatological exfoliants are those representing products and devices used in professional offices of dermatologist and estheticians utilizing chemical and abrasive forms intended to be left on the skin for limited time before removal. Over-the-counter and retail cosmetic chemical exfoliants, which can come in finished formulations such as gels, creams or lotions, can be left the skin overnight or removed shortly after application. Most, if not all liquid abrasive consisting of suspended particulate exfoliant compositions (i.e. scrub cleansers) are intended for rinse off after use.

Examples of abrasive exfoliant compositions have been cited extensively in patent art. For example, U.S. Pat. No. 6,432,430 teaches of an exfoliating scrub composition of walnut shells which is removed after application. U.S. Pat. No. 6,294,179 teaches a method of exfoliating skin using abrasive particles of a mean diameter of 40-400 micrometers and a bulk density of 1 to 4. U.S. Pat. No. 6,764,991 teaches an exfoliant toilet bar composition which utilizes physical exfoliant particles. U.S. Pat. No. 3,092,111 cites mineral particles (such as Aluminum Oxide, bentonite, diatomaceous earth) as abrasive in exfoliant paste compositions.

Many examples of chemical exfoliant compositions can be cited in the patent art using alpha hydroxyl acids, and even a combination of chemical and physical abrasive exfoliant composition can be exemplified in U.S. Pat. No. 5,939,085.

Most, if not all, physical exfoliant and powdered cosmetic compositions are not intended to be applied to the face just prior to retiring for the night because such compositions transfer from the face to a pillow or other bedding. Consequently, women normally sleep without makeup applied to their face.

Nighttime can be an advantageous time for an individual to apply exfoliant and other compositions to the face, or other epidermal areas, because the individual is not engaged in other activities and because when the body is resting it sometimes is better able advantageously to react to or to utilize certain compositions.

The compositions describe herein relates to a leave-on exfoliant powder composition that is used overnight during sleep or resting hours, or is used during the day. Furthermore, the invention described herein relates to a leave-on powdered exfoliant pigmented product that is used during daytimes or during sleep or resting hours.

An improved exfoliant composition has been discovered that can be facially applied just prior to bedtime and that generally remains on or with the users skin during sleep and, therefore, does not transfer to bedding; or, does not transfer to clothing when worn during the day. The exfoliant is also minimally irritating to the skin.

The improved exfoliant composition is in the form of a powder. Although it is possible for the power to have minimal amounts of water of hydration or other moisture, for example less than 5% by weight water, the powder preferably contains no water. The size of particles in the powder is in the range of 45 to 300 microns, preferably 50 to 200 microns. Since the powder composition contains little to no water, a relative pH can not be measured. However, a 10% aqueous dispersion of the powder composition in water provides a pH range of 5.0 to 7.0, preferably 5.5-6.5.

The powder is hygroscopic, which has a high affinity to moisture and even in dry form after application to the face, is capable of binding to moisture from the skin or from the atmosphere. This hygroscopic character is seen mostly upon application to the skin where it is freely exposed to skin and environmental moisture. Once the powder is applied to skin, the powder adsorbs moisture from the skin and the ambient air. This hygroscopic effect is capable of facilitating the manner in which other ingredients can be hydrolyzed and be utilized by skin. For example, utilization of Vitamin C and other water soluble vitamins in the powder compositions can be more readily bio-available for utilization by the skin once they are hydrolyzed by the hygroscopic nature of the powder composition.

Hygroscopic powder provides the dry exfoliating composition with a multiple-function action that progresses through multiple stages. In the first stage, when the powder is initially applied to the skin, it is dry and because of the dry particulate, can work primarily as a gentle abrasive. In the second stage, as the composition adsorbs moisture from skin or environmental air, and the moisture can interact with vitamins and other components in the composition to initiate or support enzymatic or other mechanisms that facilitate exfoliation and facilitate moisturizing the skin. One quantity of moisture can be necessary to solubilize a particular vitamin or mineral to facilitate its absorption by the skin. Another, greater, quantity of moisture has to be adsorbed to solubilize another vitamin or to moisturize noticeably the skin.

One advantage of the dry exfoliating fine powder composition of the invention is that is it relatively stable because it is in a dry state, which provides an extended shelf life. In addition, as noted, the fine powder blends into the skin, and ordinarily does not transfer to bedding while the user sleeps. Since the powder blends into and remain on the skin, colorants and other compounds found in cosmetics can be incorporated in the powder, which permits the powder to function both as cosmetic for beautifying appearance and an exfoliant.

When the dry exfoliating composition of the invention is applied daily to skin in layers of one to three, the composition can remove dead skin at a rate comparable to milder conventional exfoliation products such as products containing a low concentration of Alpha Hydroxy Acids.

In order to produce a reasonable amount of skin exfoliation, the dry exfoliating composition of the invention is applied at least once a day during evening in one to three layers for at least 30 consecutive days, preferably 60 consecutive days, and most preferably 90 consecutive days. During any such consecutive day period, missing one or two days ordinarily does not significantly adversely affect the skin exfoliation accomplished by utilizing the thy exfoliating composition of the invention.

Consequently, as used herein, it is understood that the terminology consecutive day period indicates that during one or two days in such period the exfoliating composition can not be applied.

In certain cases, uses of specific vitamins can be advantageous within the composition. In the case of Vitamin C, the anhydrous powder composition is particularly advantageous as Vitamin C is a highly unstable compound when in solution. Many references regarding the poor stabilize of Vitamin C can be cited, but as an example of a patent reference which clearly elude to the formulation challenge when using Vitamin C, U.S. Pat. No. 5,935,584 teaches a manner in which to keep the Vitamin C in separate compartment as a dry powder as part of dual system package to preserve the stability until combined with the water containing vehicle of a separate compartment upon application.

Surprisingly, the powder composition discovered and described herein is capable of exfoliating skin. Even though the composition of the invention comprises a dry powder, the composition apparently only minimally irritates the skin of a user while exfoliating on a par with many conventional exfoliating.

One important component of the improved dry powder exfoliant composition is a hygroscopic powder comprised of minerals. The mineral powder preferably includes both macro and micro minerals; preferably includes at least eight macro minerals (calcium, chlorine, magnesium, manganese, phosphorous, potassium, silicon, sodium) and sixty micro minerals; preferably has low concentrations of arsenic, lead, mercury and cadmium; and, preferably includes rare earth elements. One desirable, apparently important, component of the mineral powder is a hygroscopic powder mineral composition of the type set forth in Table 1 herein, preferably is derived from naturally occurring clay soil. The hygroscopic powder mineral composition can comprise from 0.01 to 100% by weight, preferably from 0.01 to 10% by weight, and most preferably 0.01 to 5.0% by weight of the powder exfoliant composition of the invention. The hygroscopic powder mineral composition of Table 1 adsorbs moisture, i.e., attracts and holds moisture to the surface of the powder particles. Although the powder composition of Table 1 can be utilized alone, combining it with other minerals, vitamins, herbs, etc. can improve the effectiveness of the composition as a dry skin exfoliant.

Examples of vitamins that are available in powder form and that can be utilized in the powder exfoliant composition of the invention include Vitamin A acetate, Vitamin A palmitate, thiamine mononitrate (Vitamin B1), Ribloflavin (Vitamin B2); Niacinamide (Vitamin B3); Calcium d Panthothenate (Vitamin B5); Pyrixidine Hydrochloride (Vitamin B6); Cyanocobalaimin (Vitamin B12); Ascorbic Acid (Vitamin C) and salts like sodium or calcium ascorbate; Beta Carotene; Vitamin D2 (Erogcalciferol); Vitamin D3; Vitamin E Acetate (Tocopheral acetate); and, Vitamin K. Vitamins in powder form can comprise from 0.01 to 100% by weight, preferably from 0.01 to 10% by weight, and most preferably 0.01 to 5.0% by weight of the powder exfoliant composition of the invention.

A vitamin (or the mineral composition of Table 1) in powder form can facilitate the removal of dead skin cells while providing a nutrient value that maintains or improves the health of underlying living skin, or, that helps repair any damage to underlying living skin cells.

The ascorbic acid (Vitamin C) can, if available in powder form, comprise an ester of ascorbic acid selected from the group consisting of fatty acid monoesters, fatty acid diesters, fatty acid trimesters, fatty acid tetraesters, and mixtures thereof. The ascorbic acid can, if available in powder form, comprise an ester of ascorbic acid selected from the group consisting of ascorbyl palmitate, ascorbyl laurate, ascorbyl myristate, ascorbyl stearate, ascorbyl diplamitate, ascorbyl dilaurate, ascorbyl dimyristate, ascorbyl distearate, ascorbyl tripalmiate, ascorbyl trilaurate, ascorbyl trimyristate, ascorbyl tristearate, ascorbyl tetrapalmitate, ascorbyl tetralaurate, ascorbyl tetramyristate, ascorbyl tetrastearate, and mixtures thereof.

The ascorbic acid can, if available in powder form, comprise a salt of ascorbic acid selected from the group consisting of ascorbyl phosphate, ascorbyl sulfate, and mixtures thereof.

The ascorbic acid can, if available in powder form, comprise a salt of ascorbic acid selected from the group consisting of L-ascorbic acid 3-phosphate, L-ascorbic acid 2-phosphate, L-ascorbic acid 3-pyrophosphate, his (L-ascorbic acid 3,3-) phosphate, L-ascorbic acid 3-sulfate, L-ascorbic acid 2-sulfate, L-ascorbic acid 3-pyrosulfate, bis (L-ascorbic acid 3,3-) sulfate, and mixture thereof.

In one embodiment of the powder exfoliant composition of the invention, the ascorbic acid can comprise from 0.01% to 100% by weight of the powder exfoliant composition.

An additional component of the powder exfoliant composition of the invention can consist of plant derived powdered ingredients that have been lyophilized or otherwise rendered dried. Such plant ingredients can include Aloe Barbadensis as in Aloe Vera Powdered Juice Extract, Camellia Sinensis as in Green or White Tea Powdered Extract, Rosemarinus Officinale as in Rosemary Powdered Extract, Phyllanthus Emblica as in Emblica Fruit Extract, and Triticum Vulgare as in Whet Germ Extract. The plant derived powdered extracts provide antioxidants and natural anti-inflammatory effects to the composition and their content in the powder exfoliant composition can range from 0.001% to 5% by weight, preferably from 0.001 to 1% by weight, and most preferably from 0.005% go 0.5% by weight.

Since the powdered composition has as one objective beautifying the appearance of skin during the evening resting or sleeping hours, the powder exfoliant composition can include ingredients used in color cosmetics. Such ingredients include mineral-derived powders that provide absorbency, coverage, sheen, opacity, and color. Examples of such powders are Bismuth Oxychloride, Mica, Titanium Dioxide, Iron Oxides, Zinc Stearate, Barium Sulfate, Aluminum Starch Oxtenyl Succinate, and Silica. Such cosmetic powers can, in the powder exfoliant composition, comprise 0.01% to 50% by weight, preferably 0.01% to 40% by weight, and most preferably 0.01% to 25% by weight.

The powdered exfoliant composition can be applied as desired, but typically a powder puff or other applicator is utilized to apply once daily one to three layers of the powder composition to the skin, after which the powder composition is gently massaged into the skin.

### Example 8

TABLE 9
Powder Exfoliant Composition

| | Weight |
|---|---|
| Ingredient | Percent |
| Bismuth Oxychloride (opacifiers/sheen) | 26.0 |
| Mica/Iron Oxide and Barium Sulfate (sheen/pigments) | 20.6 |
| Aluminum Strach Octenyl Succinate (binding agent) | 20.0 |
| Mica, Iron Oxide, and Titanium Dioxide (sheen/pigments) | 10.0 |
| Illite (clay) | 8.0 |
| Zinc Stearate (binding agent) | 4.0 |
| Silica (smoothing agent) | 3.0 |
| Soil Mineral Concentrate (powder) of Table 1 (exfoliant) | 2.5 |
| Ascorbic Acid (micro powder) (exfoliant; antioxidant) | 5.0 |
| Aloe Barbadensis Juice (powder form)(emollient) | 0.10 |
| Phyllanthus Embilica Fruit Extract (antioxidant; anti- | 0.50 |
| inflammatory) | |
| Dry Vitamin A Palmitate (exfoliant; antioxidant) | 0.05 |
| Tocopheryl Acetate (antioxidant) | 0.05 |
| Ectoin (antioxidant) | 0.05 |
| Cyclopia Intermedia (antioxidant) | 0.05 |
| Triticum Vulgare (Wheat) Germ Extract (antioxidant) | 0.05 |
| Rosmarinus Officialis (antioxidant) | 0.02 |
| Camellia Sinensis (antioxidant) | 0.02 |
| Aspalathus Linearis (antioxidant) | 0.01 |
| TOTAL | 100 |

### Procedure:

Admix all ingredients in a blender and pulverize to powder comprised of particles each having a maximum width in the range of 100 to 200 microns. Package powder in sealed container to prevent powder from adsorbing moisture from the air. One preferred embodiment of the invention simply comprises one or more exfoliant powders.

Another preferred embodiment of the invention simply comprises one or more pigments and one or more exfoliant powders.

A further preferred embodiment of the invention comprises pigment(s), exfoliant(s), and antioxidant(s).

Clay(s), a smoothing agent(s), an emollient(s), and/or an anti-inflammatory can also be included in the preferred embodiments of the invention to enhance the exfoliation and health of the skin.

As used herein, a therapeutically effective amount of an embodiment of the dry powder exfoliant composition of the invention is an amount sufficient to enhance and improve the normal rate of exfoliation of the skin of an individual.

### Example 9

The powder of Example 8 was clinically tested at Clinical Research Labs (CRL) in New Jersey with a 200 woman panel test for Repeat Insult Patch Testing (RIPT). Unlike most abrasive exfoliating products and chemical (Al-IA) products which can show irritation scores, the powder of Example 8 showed “0” scores across the entire study. This qualifies the powder of Example 8 as a non-irritating, exfoliation product. Although not yet clinically demonstrated, one reason the powder is believed to be non-irritating is the ability of the powder to deliver mineral nutrients to living cells in the skin and enzymatically causing exfoliation, thus providing a non-irritating pathway relative to conventional chemical or physical exfoliants.

### Example 10

A study was performed using the powder of Example 8. Thirty female subjects ranging In age from 30 to 46 years old were selected. The subjects were in general good health; refrained from taking any systemic medications during the study; refrained from use of suntan salons and excessive exposure to the sun during the study; used a normal wash routine during the study but refrained from use of washcloths, loofahs or coarse sponges on the test sites on the subjects' skin; were not pregnant or lactating and agreed to be abstinent or practice a medically acceptable form of contraception during the study; had not participated in a similar study within two weeks of the initiation of the study; and, did not have know allergies to the ingredients of the powder of Example 8.

The subjects were each given a bar of non-moisturizing soap (Aveeno™ Balancing Bar) to use for a 7-day pre-conditioning phase. Subjects were instructed to discontinue use of moisturizers, lotions, sunscreens, sking treatment products, cleansers, body scrubs, washcloths, loofahs and/or coarse sponges on the arms for the entire length of the study.

After the 7 day pre-conditioning phase was completed, three sites were identified on each subject. Two test sites (measuring 5 cm by 5 cm) were selected on each volar surface of the right and the left forearm between the elbow and the wrist, and each site was assigned 1 of 2 test material treatments. The first treatment included the powder of Example 8 alone. The second treatment included the powder of Example 8, followed by one (1) application of Cetaphil™ Daily Facial Moisturizer with SPF 15. Assignment of treatment to the right or the left forearm was determined by a computer-generated randomization code. Treatment designation remained consistent throughout the study period. For each subject, one control site was selected on either the right or the left bicep, as specified by the computer-generated randomization code. The control site remained untreated for the duration of the study.

The test and control sites of each subject were cleansed with 70% isopropyl alcohol, and treated sites were outlined with gentian violet marking pen in order to assist subjects in the location of appropriate test material application sites. The centers of the test and control sites were patched under occlusive conditions (Coverlet™ Adhesive Dressing, Beiersdorf Laboratories Inc., Norwalk, Conn.) with approximately 0.2 grams of Dansyl Chloride (5% in Petrolatum). After being patched with Dansyl Chloride, subjects were instructed to leave the patches in place for 24 hours from the time of application. Approximately 24 hours post-patching, subject returned to CRL to have the Dansyl Chloride patches removed. At this time, the test and control sites (treat and untreated) were examined use a long wavelength UV lam (Wood's Lamp, Spectroline ENF-260C, Spectronics Corporation, Westbury, N.Y.). Examination of the forearms was conducted in a darkened room.

Each site was graded for the presence of fluorescence by an evaluator that was blinded as to the treatment assignment for each site. Fluorescense was graded according to the following scale (half-point scoring was acceptable):

5—Uniform, very bright

4—Uniform, moderate brightness

3—Faded, yet visible fluorescence

2—Faded, spotty appearance

1—Complete disappearance

The day following the removal of the Dansyl Chloride patch and grading of test and control sites, subjects were given the test material and a Daily Diary, in which to record each application and any comments related to the test material use. Each subject was directed to perform specified product applications on the designated forearm site twice daily, 7 days per week. All subjects returned to Clinical Research Laboratories, Inc. daily (Monday through Friday only) for approximately three consecutive weeks. Each subject was permitted one missed visit throughout the study period. Missed visits and weekend days were assigned scores equal to the average of scores for the preceding evaluation and the evaluation following the missed visit/weekend day. Each weekend day was assigned the same average score. All subjects were instructed to return the Daily Diary as well as any remaining test product at the final visit. Fluorescence scores at each visit were recorded for all sites.

The test material application instructions consisted of the following procedures:

Treatment 1

Powder of Example 8 was applied by using a powder puff to swipe the forearm skin twice with the powder of Example 8 and then the powder was rubbed into the skin uniformly, until fully absorbed.

Treatment 2

Powder of Example 8 was applied by using a powder puff to swipe the forearm skin twice with the powder of Example 8, and then the powder rubbed into the skin uniformly, until fully absorbed. One pump of Cetphil™ Daily Moisturizer with SPF 15 was then applied and rubbed into the skin until completely absorbed.

Twenty-five subjects completed the study. Five subjects discontinued study participation for reasons unrelated to the test material.

Cumulative fluorescence scores for treated and control sites on the subjects' arms were calculated at Days 3, 5, 7, and 23.

The cumulative fluorescence scores at Day 3, Day 5, and Day 7 evidenced statistically significant differences in measures of the rate of exfoliation between Treatment 2 (powder of Example 8 with Cetphil™ Daily Moisturizer with SPR 15) and the control, demonstrating a faster rate of epidermal cell exfoliation for sites treated with Treatment 2 relative to that of untreated control sites. For cumulative fluorescence scores at Day 3, Day 5, and Day 7, there were no significant differences between Treatment I (powder of Example 8, used alone) and the control.

The cumulative fluorescence scores at Day 3, Day 5, and Day 7 evidenced statistically significant differences in measures of the rate of exfoliation between Treatments 1 and 2 and the control, demonstrating a faster rate of epidermal cell exfoliation for sites treated with Treatments 1 and 2 relative to that of untreated control sites. Each site treated was treated only with Treatment 1 or only with Treatment 2.

### Example 11

An independent controlled laboratory clinical study was performed utilizing the composition of Example 8. Of the 36 women that completed the study, over 67% of the women felt that the composition of Example 8 improved their skin by (1) improving skin texture, (2) producing smoother skin, (3) refreshing the skin, (4) revitalizing the skin, (5) producing firmer more resilient skin, (6) permitting the skin to retain more moisture, (7) producing clearer skin, (8) producing skin with fewer imperfections, (9) producing a more even skin tone, (10) producing less visible pores, (11), producing skin that feels healthier, and (12) feeling good when applied to the skin.

### Example 12

TABLE 10
Exfoliant Powder Composition
The following ingredients are provided.

| Ingredient | Weight % |
|---|---|
| Ascorbic Acid (micro Powder) | 10.0 |
| Mineral Composition of Table 1 (Powder) | 90.0 |
| TOTAL | 100.00 |

### Procedure:

All ingredients are mixed together in a blender and pulverized to powder comprised of particles each having a maximum width in the range of 100 to 200 microns (i.e., micrometers). Powder is packaged in sealed container to prevent powder from adsorbing moisture from the air. The powder is hygroscopic. The pH of an aqueous dispersion containing five percent by weight of the mineral powder in this Example 12 typically is about 3.0, and can be in the range of 3.0 to 6.5.

### Example 13

A study is performed utilizing the powder composition of Example 12. The Powder of Example 12 is applied by using a powder puff to swipe the forearm skin twice and then the product is rubbed into the skin uniformly, until fully absorbed. Of the women completing the study, over 70% felt that the composition of Example 12 improved their skin by (1) improving skin texture, (2) producing smoother skin, (3) refreshing the skin, (4) revitalizing the skin, (5) producing firmer more resilient skin, (6) permitting the skin to retain more moisture, (7) producing clearer skin, (8) producing skin with fewer imperfections, (9) producing a more even skin tone, (10) producing less visible pores, (11), producing skin that feels healthier, and (12) feeling good when applied to the skin. In some cases, the composition is applied just prior to bedtime. The composition does not, while the subject is sleeping, transfer from the skin of the subject to bedding in any manner visually noticeable to the naked eye. The nightclothes worn by each subject do not cover the forearm so that the forearm directly contacts bedding while the subject sleeps. Each of the women experiences minimal or no skin irritation when the powder was applied.

### Example 14

TABLE 11
Exfoliant Powder Composition
The following ingredients are provided.

| Ingredient | Weight % |
|---|---|
| Mineral Composition of Table 1 (Powder) | 100.00 |
| TOTAL | 100.00 |

### Procedure:

Powder is pulverized to powder comprised of particles each having a maximum width in the range of 100 to 200 microns. Powder is packaged in sealed container to prevent powder from adsorbing moisture from the air. The powder is hygroscopic. The pH of art aqueous dispersion containing five percent by weight of the mineral powder in this Example 7 typically is about 3.0, and can be in the range of 3.0 to 6.5.

### Example 15

A study is performed utilizing the powder composition of Example 14. The Powder of Example 14 is applied by using a powder puff to swipe the forearm skin twice and then the product is rubbed into the skin uniformly, until fully absorbed. Of the women completing the study, over 70% felt that the composition of Example 12 improved their skin by (1) improving skin texture, (2) producing smoother skin, (3) refreshing the skin, (4) revitalizing the skin, (5) producing firmer more resilient skin, (6) permitting the skin to retain more moisture, (7) producing clearer skin, (8) producing skin with fewer imperfections, (9) producing a more even skin tone, (10) producing less visible pores, (11), producing skin that feels healthier, and (12) feeling good when applied to the skin. In some cases, the composition is applied just prior to bedtime. The composition does not, while the subject is sleeping, transfer from the skin of the subject to bedding in any manner visually noticeable to the naked eye. The nightclothes worn by each subject do not cover the forearm so that the forearm directly contacts bedding while the subject sleeps. Each of the women experiences minimal or no skin irritation when the powder was applied.

### Example 16

Examples 12 and 13 are repeated utilizing a powder form of Vitamin A in place of the Vitamin C powder. Similar results are obtained.

### Example 17

Examples 12 and 13 are repeated utilizing a powder form of Vitamin B12 in place of the Vitamin C powder. Similar results are obtained.

### Example 18

Examples 12 and 13 are repeated utilizing, in place of the Vitamin C powder, a powder containing equal parts of powder forms of Vitamin B2, Vitamin E acetate, and Vitamin K. Similar results are obtained.

### Example 19

Examples 8 to 20 are repeated except that the powders utilized are comprised of particles having a width each in the range of about 75 to 275 microns instead of in the range of 100 to 200 microns. Similar results are obtained.

### Example 20

Example 10 is repeated except that the powder is not applied to the arm of each subject. Instead two test sites and a control site on the face of each subject are selected. Similar results are obtained.

### Example 21

Example 20 is repeated except that the powder exfoliating composition is applied facially by each individual just prior to bedtime and the composition does not, while the individual is steeping for an eight hour period, transfer from the skin of the individual to pillows or other bedding in any manner visually noticeable to the naked eye. When a powder exfoliating composition of the invention is applied to skin of an individual that is exposed to and contacts a pillow or other bedding while the individual is sleeping, then less than 80%, preferably less than 10%, more preferably less than 5%, and most preferably less than 2% by weight of the composition transfers from the skin of the subject to the bedding while the individual is sleeping for an eight hour period. The hygroscopic property of the powder composition, as well as the non-existent or low water content of the powder composition and the fine particle size of the composition are believed responsible at least in part for the non-transference of the powder from the skin of an individual to bedding.

It has been well established that oxidation or “oxidative stress” to living cells is one of the major destructive factors that works against the integrity if healthy tissue. Oxidation has also been implicated in accelerating aging and disease.

Oxidation occurs as a result of both intrinsic and extrinsic actors which initiate the formation and propaption of free radicals. Free radicals are reactive molecules which slowly attack healthy cells by targeting cell membranes and subsequently affecting normal function and structure in a negative manner. Oxidation by its own mechanism is a self propagating event that endures to further oxidation until enough damage is done to alter DNA, and or permanently destroy healthy cells.

Antioxidants are molecules or compounds which have a direct effect on free radicals by quenching or blocking the highly reactive molecule and preventing it from cascading and causing continued damage to cells.

Intrinsic oxidation factors are the result of normal metabolic function, as for example stemming from digestion of proteins, while others are chemically triggered as exemplified by free radicals generated as a result of high levels of stress. Extrinsic factors can for example range from pollution, smoking, and UV exposure.

A healthy diet of fruits and vegetables delivers natural antioxidants that suppress many of the oxidative reactions triggered by intrinsic factors. However, with the increase in extrinsic factors such as pollution and UV rays, oral supplements rich in antioxidants have become essential to fight against the ravishes of free radicals. Antioxidant supplements consist of vitamins such as vitamin E (tocopherol), minerals such as zinc and selenium, and fruit and vegetables extract rich in polyphenols.

In skin, free radicals and the important role of antioxidant in personal care products is well known to those who practice in the art. Oxidation or free radical generation can accelerate all the factor of aging and even promote skin cancer. Hence, the skin care industry practices the use of antioxidant in many formulations as a way to help skin fight against free radicals.

Unfortunately certain skin care products themselves have been known to create generation of free radicals. Among these potentially oxidizing product are skin cleansers since their propensity to irritation and inflammation is significant and both of these are interrelated with free radical formation.

The need for cleansing of skin dates back to the beginning of humankind. Use of water was the most practical means to free skin from dirt and impurities, and for reducing body malodors. It also probably represents the most gentle and least oxidizing form of cleansing.

Early forms of soap, a combination of fats, oils and salt, subsequently became popular during ancient Roman times as a method of removing bodily dirt and grime through its detergent action when combined with water. Later, goat fat, water and potassium carbonate ash created a more solid form of soap which was more practical for use in bathing. As greater advances in soap making techniques and industrialization came about, solid soap became the most popular cleansing aid during the mid 19th century. As soap evolved to modern sophistication, so did the incidence for irritation and inflammation and as a result the greater the propensity for generation of free radicals.

Facial cleansing is perhaps to the most critical form of skin cleansing from a sensitivity standpoint. Facial skin, and in particular the area surrounding and including the eyes, are highly susceptible to irritation caused by soap. In addition, facial skin seems to be more prone to sensitivity as it relates to allergies, erythematic reactions such as redness, inflammation, and dryness as it relates to cleansing products used. Because facial cleansing is also perhaps the most obvious visible site of irritation relative to other parts of the body, this also plays a role in how consumers perceive gentleness during cleaning, creating psychological sensitivity to any facial cleansing product as it relates to hygiene and beautification in a safe manner.

Facial cleansing, particularly with women, who have increased their use of cosmetics around the world throughout the last century, has become a more challengeable task for soap, as greater cleansing action requires greater use of soap, particularly at the end of the day when make-up removable is typically performed. Unfortunately, soap is not considered mild enough for facial cleansing as it can be irritating and drying, mainly due to the harshness of the nature of the surfactants, and to the pH which tends to be much above pH 7, a pH not ideally compatible with skin natural pH of 5.2-5.8. Furthermore, the higher pH of soap can be conducive to bacterial growth making it a risk to acne prone users on facial area.

Thus emollient cream cleansers, also referred to as cold creams, became very popular after the turn of the 20th century as a way to replace soap for removing dirt, grime and make-up without the irritating or drying effect on the skin.

Today's facial cleansing products have evolved to provide effective cleansing attributes with skin conditioning benefits with greatly reduced incidences of irritation. Cleansers can be found in many forms ranging from solids natural systems such as glycerin soaps, to water based single phase mild surfactant foaming products, to more emollient dual phase emulsion cleansers that can be effective in cleansing facial skin with very little foaming effect during application and rinsing. Product forms can range from liquid, liquid gels, lotions, creams, mousses, and towelletes as examples.

The invention described herein pertains to a method of producing a mineral composition described herein, and to the discovery that such mineral composition has an usually strong antioxidant effect when compared to those seen in the most popular antioxidant renowned fruits.

The antioxidant properties displayed by the mineral composition can be either hydrophilic in its' antioxidant action and specific to quenching free oxygen radicals generated, and/or lipophilic in its' antioxidant action and specific to lipid peroxidation as they effect hydrophobic cell membranes or interstitial lipid structure which makes up the cohesive and barrier function of the skin.

More specifically, a determination of oxygen radical absorbance capacity (ORAC) was conducted at Sunny Biodiscovery Laboratories in Santa Clara, Calif., to assess the mineral composition versus fruits such as blueberries, pomegranates, and cherries. The investigating lab recognized that because of its damaging effect on vital biological systems, oxidative stress has been implicated in more than 100 diseases (Ames et al., 1993). In the skin, they also recognized that oxidative stress is increased upon exposure to sunlight, pollutants and during inflammation, significantly contributing to dermal aging. Therefore, the concluded that compounds capable of shielding skin from the oxidative stress are extremely valuable for cosmetic and dermatological applications and the objective of the study was to measure the antioxidant potential of the mineral composition using oxygen radical absorbance capacity (ORAC) assay.

The test method for ORAC assay was performed according to the method described by Ou et al. (2001), with minor modifications. The ORAC assay measures the ability of antioxidant components to inhibit the decline in disodium fluorescein (FL) (Sigma-Aldrich, St Louis, Mo.) fluorescence that is induced by the peroxyl radical generator, 2′,2′-Azobis (2-amidinopropane) dihydrochloride (AAPH) (Wako Chemicals, Richmond, Va.). The reaction is conducted in the 96 well plate format. The reaction mixture contains test materials, FL (6.3×10−7M) and AAPH (1.28×101M) in phosphate-buffered saline (PBS). The reaction is started by the addition of AAPH. Fluorescence is measured at the emission length of 530 nm and excitation length of 485 nm using microplate fluorometer Cytofluor 2350 (Millipore). ORAC values are calculated from the rantification of the areas under the FL decay curve and are expressed as micromole (μmol) Trolox equivalents (TE) per g.

### Example 22

The powdered mineral composition describe herein has been found to have good antioxidant activity amounting to 320 μmole TE/g and compares favorably with fruits famous for their antioxidant activity, such as blueberries (Compound M is 15 times better gig), pomegranates (10 times better) and sour cherry (20 times better, Blando et al., 2004).

References used in these test include Ames B N, Shigensaga M K, Hagen T M. Oxidants, antioxidants, and the degenerative diseases of aging. Proc Natl Acad Sci USA 1993, 90:7915-22. Blando F, Gerard C, Nicoletti I. Sour Cherry (Prunus cerasus L) Anthocyanins as Ingredients for Functional Foods. Journal of Biomedicine and Biotechnology 2004, 5:253-258.0u B, Hampsch-Woodill M, Prior R. L. Development and validation of an improved oxygen radical absorbance capacity assay using fluorescein as the fluorescent probe. J Agric Food Chem. 2001, 49:4619-4626. The invention herein further describes methods for producing antioxidant skin cleansing compositions that includes an unusually large numbers of minerals, described herein as mineral composition. Furthermore, the antioxidant properties displayed by the large number of minerals described herein is maximized when added into solution and immediately used on skin.

In addition, the antioxidant cleansing composition is devoid of any sulfates or anionic surfactants such as sodium lauryl sulfate as these have been recognized as potential promoters of free radicals or free radical generators. Hence, the cleansing compositions described herein are sulfate-free.

The antioxidant skin cleaning composition can be applied topically to any part of the body, most particularly the area of the face. More importantly, the antioxidant cleansing compositions describe herein are designed to minimize the amount of oxidative stress that can be triggered by the action of cleansing.

The utilization of minerals as described herein and as utilized in a cleansing composition, is expected to synergistically or complimentarily has an antioxidant effect with other commonly used antioxidant ingredients to create a superior non-oxidizing antioxidant cleansing product.

The preferred embodiment of the inventions described herein describes an anhydrous, and most preferably a powdered antioxidant skin cleansing composition which preserves all antioxidant potential onto skin until the time of use.

The powdered antioxidant skin cleansing composition described herein can be applied directly to face first, and then spread with water to create the cleansing action before rinsing, or the product can be dispensed on the palm of the hand, mixed with water and then applied on the face before rinsing. Alternatively, either a dry or water pre-wetted brush can be used to apply the powdered skin cleansing composition. Hence, many packaging options can be used to house the cleansing composition, ranging from a sifter dispenser, to an open face jar with a spoon, to a jar with separate unit dose cup with a sponge or brush applicator.

Once the surface of the skin has been thoroughly cleansed with the powdered cleansing composition, rinsing with water to remove any loosened dirt, grime, and make-up is required before towel drying.

Furthermore, the invention described herein pertains to powdered skin cleansing composition that removes dirt, grime, make-up while conditioning in a skin compatible acidic pH that does not irritate dermal tissues when applied thereto.

Furthermore, the minerals described herein have been described as useful in providing exfoliation effects on skin, which compliments skin cleansing for renewing surface skin cells.

The powdered cleansing composition is preferably flow-able as a particulate ranging from a course semi-free flowing powder of 200-500 microns, to a fine free flowing powder of under 200 microns. The composition particle size can be physically modified to a desired micron size by physical means with the use of a grinder or pulverizer.

Furthermore, the powdered composition is substantially absent of water, containing less than 5% water, preferably less than 1%, most preferably less than 0.5%.

Use of antimicrobial agents or preservatives has been linked with potentially creating adverse skin reactions, such as irritation. Water is considered a medium for sustainable growth of microorganisms such as yeast, mold, and bacteria. Since the powdered cleansing composition described herein is substantially absent of water, there is a reduced need for antimicrobial agents or preservatives when compared to conventional water based cleansers. Therefore, the composition can be produced as a preservative-free or substantially free of preservatives. The presence of parabens in particular, as a class of effective preservatives widely used in personal care products mainly consisting of methylparaben, propylparaben, and butylparaben, are becoming ingredients of concern for safety. Since they are not required in the powdered cleansing composition, the powdered cleansing composition described here-in can be parabenfree.

The powdered cleansing composition delivers a pH to the skin of 4.0-6.2, preferable 5.0-6.0, most preferably 5.2-5.8 when combined with water during use which typically can be at a ratio of 20:1, preferably 10:1, most preferably 5:1 (water to cleanser ratio). A pH of 5.2-5.8 is an optimum pH for compatibility with the acid mantle of the skin.

The powdered cleansing composition is made up of a mineral based ingredient such as non-swelling and swelling clays. Kaolin, also known as aluminum silicates, is a non-swelling clay and is a known skin protectant in leave-on skin products. It is also recognized as an ideal absorbent of skin impurities. Use of kaolin in the powdered composition is preferably in a colloidal form and usage can range from levels of 1-99%, preferably 5-50%, most preferably 10-30%.

Other swelling clays such as Magnesium Aluminum Silicates can be used at concentrations of 130%, preferably 1-20%, most preferably 5-10%. Plant-derived ingredients have been used in skin care applications since ancient times. Plant derived ingredient can provide a multitude of skin care benefits ranging from soothing, anti-inflammatory, antioxidant, astringent, antimicrobial, antiseptic, cleansing, conditioning, and many other beneficial skin effects. The powdered cleansing composition described herein utilizes plant derived ingredients for a broad range of benefits to enhance the performance of the overall powdered cleansing composition.

Among the plant derived ingredients utilized in the powdered cleansing composition is Rice Flour. Rice flour possesses gentle and natural exfoliant properties and its natural adhesive properties help absorb dirt and oils from pores as well as balance skin oils. Rice flour is gluten-free and non-allergic to skin, and can be utilized at concentrations of 1-99%, preferably 5-50%, most preferably 10-30%.

Alternatively, other starchy gluten-free flours can be utilized as well. For example, Tapioca flour has the ability to mitigate greasiness and absorb oil and can be used at concentrations of 1-99%, preferably 5-50%, most preferably 10-30%.

Other plant derived wheat and whey ingredients can be used at lower concentrations ranging from 0.1% to 10%. These can include Hydrolyzed Wheat Starch, Hydrolyzed Wheat Proteins, and Whey Mineral Proteins as examples. Colloidal Oatmeal can also be used for its oil absorbing properties, anti-irritant and anti-inflammatory properties. All of these ingredients contribute to the skin conditioning attributes of the powdered cleansing composition.

Typically, a skin cleanser can contain a surfactant or detergent. For example, it is known that typical Facial cleansers can contain ingredients that reduce surface tension for facilitating the removal of Impurities present on skin. These surfactants can range in their chemical characteristics from non-ionic polysorbate 80, to strongly anionic sulfates such as Sodium Laureth Sulfate, to amphoteric surfactants such betaine derivatives. Many of these surfactants can be harsh on skin and irritating to eyes.

The powdered cleansing composition described herein is devoid of these harsh surfactants. Furthermore, the powdered cleansing composition described herein utilizes plant derived ingredients to reduce the surface tension of water to facilitate the removal of impurities present on the skin in a natural milder manner. Use of amino acid coconut derived surfactants is preferred in delivering mild surfactant qualities. Examples of these are glutamic acid coconut surfactants such as sodium cocoyl glutamate in powdered form.

More specifically, plant derived ingredients containing saponins, which are natural detergents or surfactants found in plants, are preferred. The cleansing composition can contain plant-derived ingredients such as conditioners and as cleansing agents. It is known in the art that plant contain natural surfactants in plant cells that are capable of reducing surface tension, and even foaming. Plant saponins, for example, provide excellent surface activity and can be ideal for use in cleansers.

More specifically, preferred plant derived ingredients for the powdered cleansing composition can be among the richest in saponins to aid in reduction of surface tension. Examples of such plants are Yucca schidigera and Quillaja saponaria. Yucca schidigem which is found in the southwest United States and Mexico and Quillaja saponaria which comes from the Soap bark tree native to Chile are preferred. Both plants stem from desert environments and quantitatively saponin rich.

Saponins contain a lipophilic nucleus which can be either a steroid or triterpinoid, with one or more water soluble carbohydrate side chain. In this regard, the surface activity reduction or surfactant properties of these plant components is attributed to both fat soluble and water soluble molecules all in each saponin molecule. Saponins are therefore a natural useful component in cleansing compositions to help remove impurities without the harshness of synthetic detergents. In addition, saponins, as a result of their surfactant attributes, have antimicrobial activity, and as part of their biological complexes can provide additional anti-inflammatory activity.

Since the powdered cleansing composition described herein is substantially free of water, a dry powdered form of the Yucca schidigera and the Quillaja saponaria is preferred. The preferred activity specifications for Yucca schidigera and Quillaja saponaria can range from 5.40% saponin content.

Yucca schidigera and Quillaja saponaria dried powder can be held in the powdered cleansing composition at concentrations of 0.1-25%, preferably 1-10%, and most preferably at concentrations of 2-5%. They are supplied by various vendors such as Garuda located in Lemon Cove, Calif., and Ultra Biologics located in Quebec, C-A115051.

The powdered cleansing composition described herein can also contain liquid plant derived ingredients which are either hydrophilic or lipophilic extracts or fractions as skin conditioning agents (i.e. emollients, conditioners, antioxidants, anti-inflammatory agents) or as aromatic essential oils. The plant derived ingredient can be derived from land or sea. Their usage can range from less than 20%, but preferably less than 10%, and most preferably less than 7.5%. Examples of these but not limited to are Coco nucifera. (Coconut Oil), Aloe Barbadensis Te2f Extract, Soybean Extract, Camellia senensis, and Sage Essential Oil.

The powdered cleansing composition described herein can also contain other ingredients practiced in the art of skin care such as vitamins and antioxidants. Examples of these are Vitamin A, C, E, and Beta carotene.

TABLE 12

| | |
|---|---|
| Kaolin Colloidal, USP | 50.00 |
| Rice Flour | 20.00 |
| Dry Mineral Element Composition | 10.00 |
| Yucca schidigera | 10.00 |
| Quillaja saponaria | 10.00 |

TABLE 13

| | |
|---|---|
| Kaolin Colloidal, USP | 30.00 |
| Rice Flour | 27.00 |
| Tapioca Starch | 10.00 |
| Hydrolyzed Wheat Protein | 3.00 |
| Whey Mineral Protein | 4.00 |
| Dry Mineral Element Composition | 5.00 |
| Yucca schidigera | 15.00 |
| Quillaja saponaria | 6.00 |

TABLE 14

| | |
|---|---|
| Kaolin Colloidal, USP | 20.00 |
| Rice Flour | 20.00 |
| Tapioca Flour | 10.00 |
| Magnesium Aluminum Silicate | 8.00 |
| Hydroxylpropyl Wheat Starch | 4.00 |
| Hydrolyzed Wheat Protein | 3.00 |
| Whey Mineral Protein | 4.00 |
| Collodial Oatmeal | 100 |
| Dry Mineral Element Composition | 7.00 |
| Aloe Barbadenis | 3.00 |
| Yucca schidigera | 5.00 |
| Quillaja saponaria | 3.00 |
| Coconut Oil | 3.00 |
| Tocopherol Acetate | 0.50 |
| Beta Carotene | 0.50 |
| Camellia Senensis | 0.50 |
| Beta Glucam | 0.50 |
| Soybean Extract | 0.50 |
| Sodium Cocoyl Gluatamate | 5.00 |
| Sage Oil | 0.50 |

Acne is a skin disorder caused by inflammation of the skin glands and hair follicles resulting from Infections of the bacteria P. Acne (Propionibacterium acnes) in the sebaceous gland. Acne can vary in types of skin lesions, sometimes appearing as whitehead, blackheads, pimples, blemishes, to moderate skin imperfections. There are many possible triggers to the infection (i.e. hormonal, diet, reaction to topical products, etc.), most often resulting in overproduction of sebum and other contributing factors to the clogging of pores. Excessive production of sebum for example can block skin pores to form whiteheads or blackheads on the surface of the skin, which manifest themselves as pus-filled inflammatory lesions on the skin. However, acne can be manifested in cyst and nodules that reside deeper in the skin.

Acne is most often experienced in young adolescence, most particularly during puberty. Typically, teenagers represent the vast majority of the market consuming topical over-the-counter (OTC) acne medications. These include medications consisting of active ingredients known to act as keratolytics to exfoliate the skin and minimize the potential for excess sebum production to clog pores and in secondary antibacterial or bacterial-static effect to help combat the infective P. acne. Examples of Keratolytic active ingredients used in the OTC acne market are salicylic acid and sulfur.

Aside from therapeutic leave-on products, there are numerous cleansers and soaps on the market to help from a hygienic standpoint.

In situations where OTC medications fail to bring about improvement in acne conditions, a visit to a dermatologist is usually recommended for more aggressive therapy. The therapy prescribed by dermatologists cover many active ingredients, but the most popular are retinoid therapy, such as Tretinoin, Bezoyl Peroxide, and antibiotics such as Erythromycin and Clindamycin.

Each individual acne lesion tends to diminish and heal over time. Emotionally, acne can be difficult to accept whether during adolescence or in adulthood. Not many people feel comfortable with acne lesions while they wait for the condition to undergo healing. Individuals with the condition tend to become self-conscious about the visible aspects of acne and prefer to have clear skin. Unfortunately, acne lesions also tend to leave behind temporary and sometimes permanent scarring that can last a lifetime. The more temporary scars involve color change of skin where acne lesions once existed. Hyper-pigmentation scars are the most common. These can take weeks to months to heal. The most severe permanent scarring range from ice pick scarring, those deep pitted scam with steep edges, to atrophic scars that are pitted but not as deep, to hypertrophic scars that sit on the surface of skin with a lumpy appearance.

There are treatments available for severe acne scarring. Most severe scar cases are treated by dermatologist with methods such as chemical peels and mechanical resurfacing techniques such as dermabrasion procedures.

Most men and women grow out of acne conditions as they enter adulthood in their lifetime. In some cases, teenage acne transitions on to a casual occurrence to non-existent after adulthood, while in some cases, acne can continue in its severity well into adulthood.

The casual occurrence of acne after age 21 is commonly referred to as adult acne. Adult acne usually appears less frequent with less severity than when the person experience acne during adolescence. For the most part, adult acne is typically manifested via mild blemish type lesions on the skin. These skin imperfections can last from 3-6 days, most frequently associated with menstrual or hormonal changes, stress, reaction to food, or as allergic responses to some topical product topical usage that can encourage a flare up or be classified as a comedogenic product.

As in young adulthood, particularly in women, these blemishes tend to have an emotional toll, and there is a need to cover them up or add camouflage in the form of make up. However, most make up foundation products, while capable of serving to add camouflage benefits can not effectively treat these blemishes while they hide them.

The invention described herein pertains to an anti-acne composition and method of producing the same.

Furthermore, the invention described herein pertains to an anti-acne composition that includes an unusually large number of minerals.

Specifically, the invention described herein pertains to an anti-acne composition that includes an unusually large number of minerals that have exfoliating properties.

More specifically, the invention described herein pertains to an anti-acne composition that includes a usually large number of minerals and which composition is in a powder form.

Even more specifically, the invention described herein pertains to an anti-acne composition that includes a usually large number of minerals and that is in a powder form and can be effectively used by itself or under make-up to treat acne lesions as a spot treatment.

Furthermore, the invention described herein pertains to an anti-acne composition that can treat acne lesions as a spot treatment while providing camouflaging effects.

Additionally, the invention described herein pertains to an anti-acne composition where monograph and non monograph anti-acne ingredients can be utilized.

The anti-acne composition is made up of a base of powdered amorphous ingredients used in conventional color cosmetic as fillers to provide adhesiveness to skin, sheen, and coverage. These include Talc, Zinc Stearate, Magnesium Stearate, Barium Sulfate, Titanium Dioxide, Iron Oxides, Mica or any Clay Mineral, Silica, Silicon Dioxide, Kaolin, Starch, and Bismuth Oxychloride, among others, can be utilized at concentrations of 0.1-90%.

For example, among the base ingredients Bismuth Oxychloride, a synthetic color additive which is a white odorless powder, can be used at concentrations of 0.1-90%, preferably 1-50%, most preferably 5-25%. Mother example is Mica, part of the phyllosilicate mineral or silicate class. These include Mica treatments such as Sericites, further exemplified by Mica treated with Magnesium Stearate at concentrations of 0.1-90%, preferably 1-50%, most preferably 5-25%.

The anti-acne composition can have multiple mechanisms of action in treating inflammatory and post inflammatory acne lesions. As such the composition can have comedolytic, antimicrobial, ant-infective, anti-Inflammatory, keratolytic, skin clarifying, astringent, and sebum regulation activities. Use of active ingredients can consist of any regulated active ingredient according to the FDA monograph guidelines, most particularly, those that are in powder form.

Sulfur, an ingredient in powder form, is allowed as a monograph active Ingredient in concentrations of 3-10% for OTC acne products. Sulfur is known to help in removing dead skin cells and helps in the natural healing process, as well as having antibacterial and anti-inflammatory action.

The anti-acne composition described herein can also have auxiliary non-OTC ingredients to help in the treatment of acne conditions by providing exfoliation effects. Examples of these are cosmetic forms of retinoic acid or commonly know as retinoic acid esters or alcohols forms that are considered non regulated or cosmetic acceptable ingredients.

One example is a cosmetic form of retinoic acid utilized in the composition described herein is Retinol at concentrations of 0.01 to 0.20%, preferably 0.05 to 0.15%, most preferably 0.10 to 0.15%. Retinol is the alcohol cosmetic form of Retinoic Acid, the prescription (Rx) acne medication. Typically safe use levels of Retinol are under 0.3% in order to avoid high incidence of skin Irritation. Retinol is a keratolytic and helps acne conditions by exfoliation effect as does glycolic acid and other alpha hydroxyl acids.

In the powder composition of the Invention described herein, an encapsulated 6.5% active Retinal complex know commercially as Cavamax W8 from Wacker Inc. is utilized. Cavamax is a cyclodextrintretinal powder complex designed to protect the activity of this highly unstable Vitamin A compound. In addition, the powder form allows it to be free of water incorporated In an anti-acne powder composition for slow release onto skin for milder longer lasting effect For example, use of Cavamax W8 at a concentration of 1.5% in the anti-acne composition (based on 6.5% Retinal activity) yields a 0.1% Retinal concentration in the final formulation.

Salicylic acid is a well recognized ingredient used in acne preparations for exfoliation and removing dead skin cells. Salicylic acid can be utilized in the composition described herein as an active ingredient at concentrations of 0.5-2%. Additionally, Salicylic acid natural precursor or derivatives of Salicylic acid, such as Salicylic acid esters can be also utilized in the composition described herein. For example, White Willow Bark contains salicin, which converts to salicylic acid in biological systems. The benefits of White Willow Bark provide keratolytic, anti-inflammatory and astringent benefit in the treatment of acne. Concentrations in the anti-acne composition of salicin range from 0.001 to 50%, preferably 0.01-10%, most preferably 0.15-1.5%. White Willow Bark Extract, commercially available from Amax Natrusource contains 15% Salicin, hence the usage in the anti-acne composition is from 1-10% to deliver between 0.15-1.5% salicin.

Salicylic derivatives, most particularly salicylic esters, can also contribute or be beneficial as auxiliary anti-acne ingredients in the composition. As an example, Betaine Salicylate, sold as Genti-fol SA by Arch Chemicals can be useful in the composition at concentrations between 0.001-20%, preferably 0.01-10%, most preferably 0.1-5%, and can provide some exfoliating effect

The anti-acne composition described herein can also utilize numerous auxiliary non-OTC ingredients to help in the treatment of acne conditions by providing antimicrobial and anti-inflammatory effects. Examples of these can be naturally derived Ingredients that are known for having antimicrobial and anti-inflammatory properties.

For example, Melaluca altenifolla extract (Tea Tree) has been recognized for having antimicrobial and anti-inflammatory properties. The anti-acne composition described herein can contain either or in combination of free forms or encapsulated Melaluca extract at concentrations of 0.01-10%, preferably 0.1-7.5%, most preferably 1-5%. Such combination of free and encapsulated forms of Melaluca antenifolia extract can offer immediate and sustain antimicrobial and anti-inflammatory benefits to the composition described herein. Melaluca altenifolla extract is available from several suppliers to the personal care industry, for example International Sourcing, Inc., and the encapsulated version is supplied as a cyclodextrin encapsulate as a dry powder and marketed as Melafreash T10-SLR by Actives Concepts, LLC.

Another example of an auxiliary non-OTC ingredient to help in the treatment of acne conditions by providing antimicrobial is Farnesol. Farnesol is a natural organic compound present in neroli, cyclamen, lemon grass, tuberose, rose, musk, balsam and tolu. It has natural antimicrobial and anti-infective benefits. It can be used as a liquid or a powder in the composition at concentrations pf 0.01-50%, preferably 0.1-10%, most preferably 1-5%. Farnesol is supplied by Symrise, Inc.

Yet another example of an auxiliary non-OTC ingredient is Aloe Vera (Aloe Barbedenis). Aloe has well documented topical qualities, some which have been demonstrated to be effective in the treatment of acne. Mostly known for treating burns, wounds and infections, Aloe helps in the treatment of acne by acting as a natural anti-inflammatory agent In addition, Aloe also contains lignin and saponins which gives it its skin penetrative ability and oil control, while exerting antimicrobial effect against bacteria. The usage of Aloe can be in liquid or powder form, most preferably powder at concentration between 0.001-5%.

Further examples of non-OTC auxiliary anti-acne ingredients Include Emblica Extract: Phyllanthus Emblica Fruit Extract which has been documented to display antioxidant, anti-inflammatory, and skin clarifying properties. It is useful in clarifying and soothing post breakout inflammation. Helps in reducing the appearance of acne induced dark pigmentation spots. Calcium Sodium Phosphosilicate (and) Mica sold as Actyssetm' from Engelhard Corporation contains oxides of silica, sodium, calcium and phosphorus, and has been reported to accelerate healing of wounds and have antimicrobial and anti-Inflammatory properties. Sophora Angustifolia Root Extract is a natural Anti-Acne agent, extracted and powdered from Bark of Sophora Angustifolia/Flavescens. It is reported to exert anti-bacterial and anti-Inflammatory effect, particularly effective against Propioni Bacterium Acne. Laminarghane, an Algae Extract known for with regulation of sebum and anti-inflammatory effect is yet another example. By way of exemplifying the uses of these, their concentration in the composition can be between 0.001-10%.

Use of Vitamins in anti-acne compounds can be helpful for their antioxidant or anti-acne benefits. Any Vitamin can be utilized in the composition, such as example Vitamin A, C, or E. The composition can also utilize Vitamin B3, Niacinamide, which is reported to display antibiotic benefits. Usage levels can range from 0.001-10%.

TABLE 15
Anti-Acne Compositions

| | |
|---|---|
| Ingredient | % w/w |

A

| | |
|---|---|
| 1-Bismuth Oxychloride | 29.00 |
| 2-Mica (and) Magnesium Myristate | 22.00 |
| 3-Mica, Iron Oxide, and Barium Sulfate | 14.00 |
| 4-Corn Starch Modified | 1.50 |
| 5-Dimethicone/Vinyl Dimethicone Crosspolymer and Silicate | 2.00 |
| 6-Illite | 4.50 |
| 7-Caolinitic Mineral Clay | 1.50 |
| 8-Zinc Stearate | 8.50 |
| 9-Kaolin | 1.00 |

B

| | |
|---|---|
| 10-Silica | 3.00 |
| 11-Soil Mineral Concentrate | 2.50 |
| 12-Calcium Sodium Phosphosilicate (and) Mica | 1.00 |

C

| | |
|---|---|
| 13-Tapioca Starch. | 3.00 |
| 14-Melaleuca Alternifolia Extract | 2.00 |

D

| | |
|---|---|
| 15-Sulfur Precipitated, USP | 3.00 |
| 16-Amorphous Fumed Silicone Dioxide. | 0.40 |
| 17-Farnesol, Maltodextrin, Dextrin | 1.00 |
| 18-Aloe Barbadensis Juice (Powder Form) | 030 |
| 19-Algae Extract | 0.20 |
| 20-Niacinantide (Vitamin B3) | 0.60 |

E

| | |
|---|---|
| 21-Phlyanthus emblica Fruit Extract. | 0.40 |
| 22-Melaleuca Alternifolia (Tea Tree) Leaf OH and Cyclodextrin | 1.00 |
| 23-Sephora Angustifolila Root Extract | 0.50 |
| 24-Betaine Salicylate | 1.50 |
| 25-White Willow Bark/15% | 3.30 |
| 26-Cyclodextrin/Retinol | 1.50 |
| TOTAL | 100 |

The method or procedure for producing the composition involves mixing each pre-mix of Phase A through E and then blending all phases together using a conventional blender. The composition can include one, two, three, four, five, six, seven, eight, nine, or all of the compositions from each category A-E. Alternatively, not all categories need to be represented, such that the compositions can comprise A-E, A-D, A-C, A-B, or just A, B-E, B-D, B-C, or just B, C-E, C-D, or just C, D-E or just D, or just E, or A and C, or A and D or A and E, or B and D or B and E, or C and E. A pulverizer can be used to create a fine particle powder.

The anti-acne composition is applied as a spot treatment wherever a blemish appears as often as needed, but preferably 2-3 times per day.

The composition was clinically tested by Clinical Research Laboratories located In Piscataway, N.J. on approximately 40 women, age 18-55 ehibiting acne lesions.

The following treatment effects and product attributes of test material were assessed favorably in statistically significant proportions (Za1.96) of the study population via consumer perception questionnaires at Week 4 are listed below:

The product feels good on the skin

The product feels gentle and natural on the skin.

Blemishes looked less red and inflamed after just one use.

Blemishes looked less red and inflamed with continued use.

Blemishes healed after two to three days of use.

Skin texture Improved.

Skin looks clearer.

Pores look smaller and more refined.

Subjects see a reduction in blackheads.

Subjects see a reduction in blemishes and breakouts.

The skin looks and feels healthier.

Overall complexion has noticeably improved.

TABLE 16
Placebo Formulation

| | | | % |
|---|---|---|---|
| INGREDIENTS | C.T.F.A. NAME | SUPPLIERS | w/w |
| Phase A | A | A | |
| 1. Biron MTU | 1. Bismuth Oxychloride | EMD | 24.7 |
| 2. Sericite O/MM3 | 2. Mica (and) Manesium | KOBO | 27.0 |
| | Myristate | | 0 |
| 3. Low Luster Pigment | 3. Mica, Iron Oxide, and Barium | EMD | 17.3 |
| | Sulfate | | 0 |
| 4. Dry Flo AF | 4. Corn Starch Modified | Nat. Starch | 1.80 |
| 5. D.C. Cosmetic Powder | 5. Dimethicone/Vinyl | Dow Corning | 2.40 |
| | Dimethicone Crosspolymer | | |
| | (and) Silica | | |
| 6. Ventilated Green Clay | 6. Illite | Tri-K Ind. | 5.30 |
| 7. Amazonian White Clay | 7. Caolinitic Mineral Clay | Jarchem | 1.80 |
| | | Industries | |
| 8. Zinc Stearate FGK | 8. Zinc Stearate | Acme- | 10.7 |
| | | Hardesty Co. | 0 |
| 9. Ochre Red Clay | 9. Kaolin | Alban Muller | 1.20 |
| Phase B | Phase B | Phase B | |
| 10. Spheron P-1500 | 10. Silica | Presperse | 3.70 |
| 11. Micropulverized Totala 72 ™ | 11. Soil Meneral Concentrate | Biokool LLC | NA |
| 12. Actyase Premiere BG | 12. Calcium Sodium | Engelhard | NA |
| | Phosphosilicate (and) Mica | | |
| Phase C | Phase C | Phase C | |
| 13. Natrosorb Bath | 13. Tapioca Starch | National | 3.60 |
| | | Starch | |
| 14. Tea Tree Oil | 14. Melaleuca Alternifolia Extract | International | NA |
| | | Sourcing Inc. | |
| Phase D | Phase D | Phase D | |
| 15. Sulfur Precipitated, USP | 15. Sulfur Precipitate, USP | Mutcher | NA |
| 16. Aerosil A 200 | 16. Amorphous Fumed Silicone | Deassa | 0.50 |
| | Dioxide | Corporation | |
| 17. Farnesol Powder | 17. Farnesol, Maltodextrin, | Symrise | NA |
| | Dextrin | | |
| 18. RitaLoe200M | 18. Aloe Vardensia Juice (Powder | RITA | NA |
| | Form) | | |
| 19. Laminarghane | 19. Algae Extract | Presperse | NA |
| 20. Niacinamide | 20. Niacinamide (Vitamin B3) | AnMar | NA |
| | | International | |
| Phase E | Phase E | Phase E | |
| 21. Emblica Extract | 21. | EMD | NA |
| 22. Melafresh T10-SLR | 22. Melaleuca Altenifolia (Tea | Active | NA |
| | Tree) Leaf Oil and | Concepts, LLC | |
| | Cyclodextrin | | |
| 23. Sophora Powder | 23. Sophora Angustifolia Root | Presperse | NA |
| | Extract | | |
| 24. Genti-Fol SA | 24. Betaine Salicylate | Arch | NA |
| 25. White Willow Bark 15% | 25. White Willow Bark/15% | Amax | NA |
| | | NutraSource, | |
| | | Inc. | |
| 26. Cavamax W8 Retinol- | 26. Cyclodextrin/Retinol | Wacker | NA |
| Complex | | | |
| | | Total | 100 |

What is claimed is:

1. A method of producing a composition for treating acne, the method comprising:
combining at least two components of each individual phase, wherein the components of Phase A are:
(a) bismuth oxychloride,
(b) mica with magnesium myristate,
(c) mica with iron oxide and barium sulfate,
(d) corn starch modified,
(e) dimethicone/vinyl dimethicone crosspolymer with silicate,
(f) illite,
(g) caolinitic mineral clay, and
(h) zinc estrum;
the components of Phase B are:
(a) kaolin,
(b) silica,
(c) soil mineral concentrate, and
(d) calcium sodium phosphosilicate with mica;
the components of Phase C are:
(a) tapioca starch, and
(b) melaleuca alterniflolia extract;
the components of Phase D are:
(a) sulfur precipitated, USP,
(b) amorphous fumed silicone dioxide,
(c) farnesol, maltodextrin, dextrin,
(d) aloe barbadensis juice in powder form,
(e) algae extract, and
(f) niacinamide;
and the components of Phase E are:
(a) phyllanthus emblica fruit extract,
(b) melaleuca altenifolia leaf oil and cyclodextrin,
(c) sephora angustifolia root extract,
(d) betaine salicylate,
(e) white willow bark, and
(f) cyclodextrin/retinol;
and once at least two components of each individual phase are mixed together, then combining all of Phases A-E together, thereby producing a composition for treating acne.

2. The method of claim 1, wherein the components of each phase are mixed in a blender.

3. The method of claim 1, wherein the phases are combined together in a blender.

4. The method of claim 1, wherein the composition is a powder.

5. The method of claim 1, wherein all of the components of each phase are used in the composition.

6. A composition produced by the method of claim 1.

7. A method of treating a subject with acne, the method comprising administering to the subject the composition of claim 6.

8. The method of claim 7, wherein the acne treating composition can be applied in spot treatments.

9. The method of claim 7, wherein the acne treating composition can be applied to an entire affected area.

10. The method of claim 7, wherein the acne treating composition can further comprise a makeup base so that it acts as a camouflage.

11. A kit comprising the composition of claim 6.

12. The composition of claim 6, further comprising other composition known for treating acne.

13. The composition of claim 6 in a pharmaceutical composition.
